(19) United States
(12) Patent Application Publication
(10) Pub. No.: US 2018/0346997 A1
(43) Pub. Date: Dec. 6, 2018

(54) METHODS AND SNP DETECTION KITS FOR PREDICTING PALM OIL YIELD OF A TEST OIL PALM PLANT

(71) Applicant: SIME DARBY PLANTATION BERHAD, Petaling Jaya (MY)

(72) Inventors: Chee Keng Teh, Selangor (MY); Ai Ling Ong, Selangor (MY); Qi Bin Kwong, Selangor (MY); Sukganah Apparrow, Selangor (MY); Mohaimi Mohamed, Selangor (MY); Fook Tim Chew, National University of Singapore (SG); David Appleton, Selangor (MY); Harikrishna Kulaveerasingam, Selangor (MY)

(21) Appl. No.: 15/552,190

(22) PCT Filed: Jul. 16, 2015
(86) PCT No.: PCT/MY2015/000061

§ 371 Date: Aug. 18, 2017

(30) Foreign Application Priority Data
Feb. 18, 2015 (MY) PI2015700516

(51) Int. Cl. C12Q 1/6895

(52) U.S. Cl. CPC C12Q 1/6895; C12Q 2600/156; C12Q 2600/13

(57) ABSTRACT

Methods for predicting palm oil yield of a test oil palm plant are disclosed. The methods comprise determining, from a sample of a test oil palm plant of a population, at least a first SNP genotype, corresponding to a first SNP marker, located in a first QTL for a high-oil-production trait and associated, alter stratification and kinship correction, with the high-oil-production trait with a genome-wide-log₁₀(p-value) of at least 4.0 in the population or having a linkage disequilibrium r¹ value or at least 0.2 with respect to a first other SNP marker linked thereto and associated, alter stratification and kinship correction, with the high-oil-production trait with a genome-wide-log₁₀(p-value) of at least 4.0 in the population. The methods also comprise comparing the first SNP genotype to a corresponding first reference SNP genotype and predicting palm oil yield or the test plant based on extent or matching of the SNP genotypes.

### TECHNICAL FIELD

This application relates to methods for predicting palm oil yield of a test oil palm plant, and more particularly to methods for predicting palm oil yield of a test oil palm plant comprising determining, from a sample of a test oil palm plant of a population of oil palm plants, at least a first single nucleotide polymorphism (SNP) genotype of the test oil palm plant, the first SNP genotype corresponding to a first SNP marker, comparing the first SNP genotype of the test oil palm plant to a corresponding first reference SNP genotype indicative of the high-oil-production trait in the same genetic background as the population, and predicting palm oil yield of the test oil palm plant based on the extent to which the first SNP genotype of the test oil palm plant matches the corresponding first reference SNP genotype, as well as SNP detection kits for predicting palm oil yield of a test oil palm plant in accordance with such methods.,

### BACKGROUND ART

The African oil palm Elaeis guineensis Jacq. is an important oil-food crop. Oil palm plants are monoecious, i.e. single plants produce both male and female flowers, and are characterized by alternating series of male and female inflorescences. The male inflorescence is made up of numerous spikelets, and can bear well over 100,000 flowers. Oil palm is naturally cross-pollinated by insects and wind. The female inflorescence is a spadix which contains several thousands of flowers borne on thorny spikelets. A bunch carries 500 to 4,000 fruits. The oil palm fruit is a sessile drupe that is spherical to ovoid or elongated in shape and is composed of an exocarp, a mesocarp containing palm oil, and an endocarp surrounding a kernel.

Oil palm is important both because of its high yield and because of the high quality of its oil. Regarding yield, oil palm is the highest yielding oil-food crop, with a recent average yield of 3.67 tonnes per hectare per year and with best progenies known to produce about 10 tonnes per hectare per year. Oil palm is also the most efficient plant known for harnessing the energy of sunlight for producing oil. Regarding quality, oil palm is cultivated for both palm oil, which is produced in the mesocarp, and palm kernel oil, which is produced in the kernel. Palm oil in particular is a balanced oil, having-almost equal proportions of saturated fatty acids (≈55% including 45% of palmitic acid) and unsaturated fatty acids (≈45%), and it includes beta carotene. The palm kernel oil is more saturated than the mesocarp oil. Both are low in free fatty acids. The current combined output of palm oil and palm kernel oil is about 50 million tonnes per year, and demand is expected to increase substantially in the future with increasing global population and per capita consumption of oils and fats.

Although oil palm is the highest yielding oil-food crop, current oil palm crops produce well below their theoretical maximum, suggesting potential for improving yields of palm oil through improved selection and identification of high yielding oil palm plants. Conventional methods for identifying potential high-yielding palms, for use in crosses to generate progeny with higher yields as well as for commercial production of palm oil, require cultivation of palms and measurement of production of oil thereby over the course of many years, though, which is both time and labor intensive. Moreover, the conventional methods are based on direct measurement of oil content of sampled fruits, and thus result in destruction of the sampled fruits. In addition, conventional breeding techniques for propagation of oil palm for oil production are also time and labor intensive, particularly because the most productive, and thus commercially relevant, palms exhibit a hybrid phenotype which makes propagation thereof by direct hybrid crosses impractical. Quantitative trait loci (also termed QTL) marker programs based on linkage analysis have been implemented in oil palm with the aim of improving upon conventional breeding techniques, as taught for example by Billotte et al. Theoretical & Applied Genetics 120:1673-1687 (2010). Linkage analysis is based on recombination observed in a family within recent generations and often identifies poorly localized QTLs for complex phenotypes, though, and thus large families are needed for better detection and confirmation or QTLs, limiting practicality of this approach for oil palm. QTL marker programs based on association analysis for the purpose of identifying candidate genes may be a possibility for palm too, as discussed for example by Ong et, al, WO2014/129885, with respect to plant height. A focus on identifying candidate genes may be of limited benefit in the context of traits that are determined by multiple genes though, particularly genes that exhibit low penetrance with respect to the trait. QTL marker programs based on genome-wide association studies have been carried out in human and rice, among others, as taught by Hirota et al., Nature Genetics 44:1222-1226 (2012), and Huang et al., Nature Genetics 42:961-967 (2010), respectively. Application of this approach to oil palm has not been practical, though, because commercial palms tend to be generated from genetically narrow breeding materials. Accordingly, a need exists to improve oil palm through improved methods for predicting palm oil yields of oil palm plants.

### DISCLOSURE OF INVENTION

In one example embodiment, a method for predicting palm oil yield of a test oil palm plant is disclosed. The method comprises a step of (i) determining, from a sample of a test oil palm plant of a population of oil palm plants, at least a first single nucleotide polymorphism (SNP) genotype of the test oil palm plant. The first SNP genotype corresponds to a first SNP marker. The first SNP marker is located in a first quantitative trait locus (QTL) for a high-oil-production trait. The first SNP marker also is associated, after stratification and kinship correction, with the high-oil-production trait with a genome-wide −log₁₀(p-value) of at least 4.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 4.0 in the population. The method also comprises a step of (ii) comparing the first SNP genotype of the test oil palm plant to a corresponding first reference SNP genotype indicative of the high-oil-production trait in the same genetic background as the population. The method also comprises a step of (iii) predicting palm oil yield of the test oil palm plant based on the extent to which the fast SNP genotype of the test oil palm plant matches the corresponding first reference SNP genotype. The first QTL is a region of the oil palm genome corresponding to one of:
(1) QTL region 1, extending from nucleotide 66542323 to 66776312 of chromosome 1;
(2) QTL region 2, extending from nucleotide 66807385 to 67299617 of chromosome 1;
(3) QTL region 3, extending from nucleotide 62277032 to 62355782 of chromosome 2;
(4) QTL region 4, extending from nucleotide 31132787 to 31173962 of chromosome: 4;
(5) QTL region 5, extending from nucleotide 32863621 to 32964104 of chromosome 5;
(6) QTL region 6, extending from nucleotide 33355931 to 33509217 of chromosome 5;
(7) QTL region 7, extending from nucleotide 33658904 to 34233352 of chromosome 5;
(8) QTL region 8, extending from nucleotide 34358119 to 34997228 of chromosome 5;
(9) QTL region 9, extending from nucleotide 35004388 to 35125743 of chromosome 5;
(10) QTL region 10, extending from nucleotide 35191678 to 35193677 of chromosome 5;
(11 ) QTL region 11, extending from nucleotide 36108847 to 36272808 of chromosome 5;
(12) QTL region 12, extending from nucleotide 39210662 to 39225076 of chromosome 5;
(13) QTL region 13, extending from nucleotide 39518005 to 40469897 of chromosome 5;
(14) QTL region 14, extending from nucleotide. 40535309 to 40690150 of chromosome 5;
(15) QTL region 15, extending from nucleotide 40789706 to 40983955 of chromosome 5;
(16) QTL region 16, extending from nucleotide 41001085 to 41302446 of chromosome 5;
(17) QTL region 17, extending from nucleotide 3050807 to 3241977 of chromosome 8;
(18) QTL region 18, extending from nucleotide 5354764 to 5445890 of chromosome 8;
(19) QTL region 19, extending from nucleotide 29488933 to 29602300 of chromosome 9;
(20) QTL region 20, extending from nucleotide 4797284 to 5717606 of chromosome II; or
(21) QTL region 21, extending from nucleotide 8611715 to 8857914 of chromosome 15.

In another example embodiment, a SNP detection kit for predicting palm oil yield of a test oil palm plant is disclosed. The kit comprises (i) a set of at least 21 nucleotide molecules suitable for determining, from a sample of a test oil palm plant of a population of oil palm plants, a first SNP genotype to a twenty-first SNP genotype, respectively, of the test oil palm plant, the first SNP genotype to the twenty-first SNP genotype corresponding to a first SNP marker to a twenty-first SNP marker, respectively, the first SNP marker to the twenty-first SNP marker (a) being located in a first QTL to a twenty-first QTL, respectively, for as high-oil-production trait in the population and (b) being associated, after stratification and kinship correction, with the high-oil-production trait with a genuine-wide −log₁₀(p-value) of a least 4.0 in the population or having linkage disequilibrium r² values of at least 0.2 with respect to a first other SNP marker to a twenty-first other SNP marker, respectively, that are linked thereto and associated, after stratification and kinship correction, with the high-oil-production trait with a genome-wide −log₁₀(p-value) of at least 4.0 in the population. The kit also comprises (ii) a reference sample of a reference high-oil-yielding oil palm plant of the population. The first QTL to the twenty-first QTL are regions of the oil palm genome corresponding, respectively, to QTL regions 1 to 21, as described above.

### BRIEF DESCRIPTION OF DRAWINGS

FIG. 1 shows quartile-quartile (Q-Q) plots of observed −log₁₀(p-values) versus expected −log₁₀(p-values) for genome-wide association studies (also termed GWAS) based on a naive model in (a) a Deli dura x AVROS pisifera population and (b) a Nigerian dura x AVROS pisifera population.

FIG. 2 shows (a, b) Q-Q plots of observed −log₁₀(p-values) versus expected −log₁₀(p-values) for GWAS and (c, d) Manhattan plots, ail based on a compressed mixed linear model (also termed MLM), in (a, c) a Deli dura x AVROS pisifera population and (b, d) a Nigerian dura x AVROS pisifera population.

FIG. 3 is an illustration of an approach for defining a range of a QTL region according to a linkage disequilibrium r² value of at least 0.2 as threshold, wherein the highlighted range is the selected QTL region in accordance with the method of predicting palm oil yield of a test oil palm plant.

FIG. 4 is a graph showing the SNP effects of an exemplary SNP, SD_SNP_000019529, as determined in a Deli dura x AVROS pisifera population and a Nigerian dura x AVROS pisifera population.

### BEST MODE FOR CARRYING OUT THE INVENTION

The application is drawn to methods and SNP detection kits for predicting palm oil yield of a test oil palm plant. The methods comprise steps of (i) determining, from a sample of a test oil palm plant of a population of oil palm plants, at least a first single nucleotide polymorphism (SNP) genotype of the test oil palm plant, (ii) comparing the first SNP genotype of the test oil palm plant to a corresponding first reference SNP genotype indicative of the high-oil-production trait in the same genetic background as the population, and (iii) predicting palm oil yield of the test oil palm plant based on the extent to which the first SNP genotype of the test oil palm plant matches the corresponding first reference SNP genotype. The first SNP genotype corresponds to a first SNP marker. The first SNP marker is located in a first quantitative trait locus (QTL) for a high-oil-production trait. The first SNP marker also is associated, after stratification and kinship correction, with the high-oil-production trait with a genome-wide −log₁₀(p-value) of at least 4.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 4.0 in the population. The first QTL is a region of the oil palm genome corresponding to one of QTL regions l to 21, as described in more detail below. Similarly, the SNP detection kits comprise (i) a set of at least 21 nucleotide molecules suitable for determining, from a sample of a test oil palm plant of a population of oil palm. plants, a first SNP genotype to a twenty-first SNP genotype, respectively, of the test oil palm plant, as described above, and (ii) a reference sample of a reference high-oil-yielding oil palm plant of the population.

By conducting genome resequencing and genome-wide association studies of oil palm plants from a semi-wild oil palm population and a commercially relevant oil palm population, including application of stratification and kinship correction, it has been determined that SNP markers that are located in 21 QTL regions of the oil palm genome and that are associated, after stratification and kinship correction, with a high-oil-production trait can be used to achieve 50% accuracy correlation and 30% accuracy correlation, respectively, in the two populations. Without wishing to be hound by theory, it is believed that identification of the 21 QTL regions and SNP markers therein that are associated, after stratification and kinship correction, with the high-oil-production trait will enable more rapid and efficient selection of candidate agricultural production palms and candidate breeding palms, tram among the semi-wild and commercially relevant oil palm populations and others. Stratification and kinship correction reduce false positive signals due to recent common ancestry of small groups of individuals within the population of oil palm plants from which a test oil palm plant is sampled, thereby making practical the method for predicting palm oil yield of a test oil palm plant based on association. The methods and SNP detection kits will enable identification of potential high-yielding palms, for use in crosses to generate progeny with higher yields and for commercial production of palm oil, without need for cultivation of the palms to maturity, thus bypassing the need for the time and labor intensive cultivations and measurements, the destructive sampling of fruits, and the impracticality of direct hybrid crosses that are characteristic of conventional approaches. For example, the methods and SNP detection kits can be used to choose oil palms plants for germination, cultivation in a nursery, cultivation for commercial production of palm oil, cultivation for further propagation, etc., well before direct measurement of palm oil production by the test oil palm plant could be accomplished. Also for example, the methods and SNP detection kits can be used to accomplish prediction of palm oil yields with greater efficiency and/or less variability than by direct measurement of palm oil production. The methods and SNP detection kits can be used advantageously with respect to even a single SNP, given that improvements in oil palm yield that seem small on a percentage basis still can have a dramatic effect on overall palm oil yields, given the large scale of commercial cultivations. The methods and SNP detection kits also can be used advantageously with respect to combinations of two or more SNPs, e.g, a first SNP genotype and a second SNP genotype, or a first SNP genotype to a twenty-first SNP genotype, given additive and/or synergistic effects.

The terms “high-oil-production trait,” “high yield,” “high-yielding,” and “oil yield,” as used with respect to the methods and kits disclosed herein, refer to yields of palm oil in mesocarp tissue of fruits of palm oil plants.

The singular forms “a,” “an,” and “the” are intended to include the plural forms as well, unless the context clearly indicates otherwise.

As noted above, a method for predicting palm oil yield of a test oil palm plant is disclosed. The method, comprises a step of (i) determining, from a sample of a test oil palm plant of a population of oil palm plants, at least a first single nucleotide polymorphism (also termed SNP) genotype of the test oil palm plant.

The SNP genotype of the test oil palm plant corresponds to the constitution of SNP alleles at a particular locus, or position, on each chromosome in which the locus occurs in the genome of the test oil palm plant. A SNP is a polymorphic variation with respect to a single nucleotide that occurs at such a locus on a chromosome. A SNP allele is the specific, nucleotide present at the locus on the chromosome. For oil palm plants, which are diploid and which thus inherit one set of maternally derived chromosomes and one set of paternally derived chromosomes, the SNP genotype corresponds to two SNP alleles, one at the particular locus on the maternally derived chromosome and the other at the particular locus on the paternally derived chromosome. Each SNP allele may be classified, for example, based on allele frequency, e.g. as a major allele (A) or a minor allele (a). Thus, for example, the SNP genotype can correspond to two major alleles (A/A), one major allele and one minor allele (A/a), or two minor alleles (a/a).

The test oil palm plant can be an oil palm plant in any suitable form. For example, the test oil palm plant can be a seed, a seedling, a nursery phase plant, an immature phase plant, a cell culture plant, a zygotic embryo culture plant, or a somatic tissue culture plant. Also for example, the test oil palm plant can be a production phase plant, a mature palm, a mature mother palm, or a mature pollen donor.

A test oil palm plant in the form of a seed, a seedling, a nursery phase plant, an immature phase plant, a cell culture plant, a zygotic, embryo culture plant, or a somatic tissue culture plant is in a form that is not yet mature, and thus that is not yet producing palm oil in amounts typical of commercial production, if at all. Accordingly, the method as applied to a test oil palm plant in such a form can be used to predict palm oil yield of the test oil palm plant before the test oil palm plant has matured sufficiently to allow direct measurement of palm oil production by the test oil palm plant during commercial production.

A test oil palm plant in the form of a production phase plant, a mature palm, a mature mother palm, or a mature pollen donor is in a form that is mature. Accordingly, the method as applied to a test oil palm plant in such a form can be used to predict palm oil yield of the test oil palm as an alternative to direct measurement of oil palm yield.

The population of oil palm plants from which the test oil palm plant is sampled can comprise any suitable population of oil palm plants. The population can be specified in terms of fruit type and/or identity of the breeding material from which the population was generated.

In this regard, fruit type is a monogenic trait in oil palm that is important with respect to breeding and commercial production. Oil palms with either of two distinct fruit types are generally used in breeding and seed production through crossing in order to generate palms for commercial production of palm oil, also termed commercial planting materials or agricultural production plants. The first fruit type is dura (genotype: sh+ sh+), which is characterized by a thick shell corresponding to 28 to 35% of the fruit by weight, with no ring of black fibres around the kernel of the fruit. For dura fruits, the ratio of mesocarp to fruit varies from 50 to 60%, with extractable oil content in proportion to bunch weight of 18 to 24%. The second fruit type is pisifera (genotype: sh− sh−), which is characterized by the absence of a shell, the vestiges of which are represented by a ring of fibres around a small kernel. Accordingly, for pisifera fruits, the ratio of mesocarp to fruit is 90 to 100%. The ratio of mesocarp oil to bunch is comparable to the dura at 16 to 28%. Pisiferas are however usually female sterile as the inajority of bunches abort at an early stage of development.

Crossing dura and pisifera gives rise to palms with a third fruit type, the tenera (genotype: sh+ sh−). Tessera fruits have thin shells of 8 to 10% of the fruit by weight, corresponding to a thickness of 0.5 to 4 mm, around which is a characteristic ring of black fibres. For tenera fruits, the ratio of mesocarp to fruit is comparatively high, in the range of 60 to 80%. Commercial tenera palms generally produce more fruit bunches than duras, although mean bunch weight is lower. The ratio of mesocarp oil to bunch is in the range of 20 to 30%, the highest of the three fruit types, and thus tenera are typically used as commercial planting materials.

Identity of the breeding material can be based on the source and breeding history of the breeding material. Dura palm breeding populations used in Southeast Asia include Serdang Avenue, Ulu Remis (which incorporated some Serdang Avenue material), Johor Labis, and Elmina estate, including Deli Dumpy, all of which are derived from Deli dura. Pisifera breeding populations used for seed production are generally grouped as Yangambi, AVROS, Binga and URT. Other dura and pisifera populations are used in Africa and South America.

Oil palm breeding is primarily aimed at selecting for improved parental dura and pisifera breeding stock palms for production of superior tenera commercial planting materials. Such materials are largely in the form of seeds although the use of tissue culture for propagation of clones continues to be developed. Generally, parental dura breeding populations are generated by crossing among selected dura palms. Based on the monogenie inheritance of fruit type, 100% of the resulting palms will be duras. After several years of yield recording and confirmation of bunch and fruit characteristics, duras are selected for breeding based on phenotype. In contrast, pisifera palms are normally female sterile and thus breeding populations thereof must be generated by crossing among selected teneras or by crossing selected teneras with selected pisiferas. The tenera x tenera cross will generate 25% duras, 50% teneras and 25% pisiferas. The tenera x pisifera cross will generate 50% teneras and 50% pisiferas. The yield potential of pisiferas is then determined indirectly by progeny testing with the elite duras, i.e. by crossing duras and pisiferas to generate teneras, and then determining yield phenotypes of the fruits of the teneras over time. From this, pisiferas with good general combining ability are selected based on the performance of their tenera progenies. Intercrossing among selected parents is also carried out with progenies being carried forward to the next breeding cycle. This allows introduction of new genes into the breeding programme to increase genetic variability.

Oil palm cultivation for commercial production of palm oil can be improved by use of the superior tenera commercial planting materials. Priority selection objectives include high oil yield per unit area in terms of high fresh fruit bunch yield and high oil to bunch ratio (thin shell, thick mesocarp), high early yield (precocity), and good oil qualities, among other traits, Progeny plants may be cultivated by conventional approaches, e.g. seedlings may be cultivated in polyethylene bags in pre-nursery and nursery settings, raised for about 12 months, and then planted as seedlings, with progeny that are known or predicted to exhibit high yields chosen for further cultivation, among other approaches.

Accordingly, in some examples the population of oil palm plants can comprise a Nigerian dura x AVROS pisifera population, a Deli dura x AVROS pisifera population, or a combination thereof. Also in some examples the population of oil palm plants comprises a Nigerian dare x Nigerian dura population, a Nigerian dura x Deli dura population, a Deli dura x Deli dura population, an AVROS pisifera x AVROS tenera population, an AVROS tenera x AVROS tenera population, or a combination thereof.

The sample of the test oil palm plant can comprise any organ, tissue, cell, or other part of the test oil palm plant that includes sufficient genomic DNA of the test oil palm plant to allow for determination of one or more SNP genotypes of the test oil palm plant, e.g. the first SNP genotype. For example, the sample can comprise a leaf tissue, among other organs, tissues, cells, or other parts. As one of ordinary skill will appreciate, determining, from a sample of a test oil palm plant, one or more SNP genotypes of the test oil palm plant, is necessarily transformative of the sample. The one or more SNP genotypes cannot be determined, for example, merely based on appearance of the sample. Rather, determination of the one or more SNP genotypes of the test oil palm plant requires separation of the sample from the test oil palm plant and/or separation of genomic DNA from the sample.

Determination of the at least first SNP genotype can be carried out by any suitable technique, including, for example, whole genome resequencing with SNP calling, hybridization-based methods, enzyme-based methods, or other post amplification methods, among others.

The first SNP genotype corresponds to a first SNP marker. A SNP marker is a SNP that can be used in genetic mapping.

The first SNP marker is located in a first quantitative trait locus (also termed QTL) for a high-oil-production trait. A QTL is a locus, extending along a portion of a chromosome, that contributes in determining a phenotype of a continuous character, i.e. in this case, the high-oil-production trait.

The high-oil-production trait relates to a trait of production of palm oil by the test oil palm plant upon reaching a mature state, e.g. reaching production phase, and upon being cultivated under conditions suitable for production of palm oil in a high amount, e.g. commercial cultivation, in an amount that is higher than average, with respect to the population of oil palm plants from which the test oil palm plant is sampled, also upon reaching a mature state and upon being cultivated under conditions suitable for production of palm oil in a high amount.

Considering a test oil plant that is a tenera oil palm plant, the high-oil-production trait can correspond, for example, to production of palm oil at greater than 3.67 tonnes of palm oil per hectare per year, i.e. above recent average yields for typical oil palm plants used in commercial production, which also are tenera oil palm plants, as discussed above. The high-oil production trait also can correspond, for example, to production of palm oil at greater than 10 tonnes of palm oil per hectare per year, i.e. above recent average yields for current best-progeny oil palm plants used in commercial production. The high-oil production trait also can correspond, for example, to production of palm oil at greater than 4, 5, 6, 7, 8, or 9 tonnes of palm oil per hectare per year, i.e. above yields that are intermediate between the recent average yields noted above. Considering a test oil palm plant that is a dura oil palm plant or a pisifera oil palm plant, the high-oil production trait can correspond to production of palm oil in correspondingly lower amounts, consistent with lower average yields obtained for dura and pisifera oil palm plants relative to tenera oil palm plants.

The high-oil-production trait can comprise increased oil-to-dry mesocarp (also termed O/DM). As noted above, palm oil is produced in the mesocarp of the oil palm fruit. O/DM is a measure of palm oil yield. Accordingly, a relatively high O/DM is an indicator of relatively high production of palm oil.

The first SNP marker is associated, after stratification and kinship correction, with the high-oil-production trait with a genome-wide −log₁₀(p-value) of at least 4.0 in the population or has a linkage disequilibrium r² value of at least 0.2 with respect to a first other SNP marker that is linked thereto and associated, after stratification and kinship correction, with the high-oil-Production trait with a genome-wide −log₁₀(p-value) of at least 4.0 in the population.

A first SNP marker being, associated, after stratification and kinship correction, with a trait with a genome-wide −log₁₀(p-value) of at least 4.0 in a population indicates that a high likelihood exists that the first SNP maker and the trait are linked.

A p-value is the probability of observing a test statistic, in this case relating to association of a SNP marker, e.g. the first SNP marker or the first other SNP marker, and the high-oil-production trait, equal to or greater than a test statistic actually observed, if the null hypothesis is true and thus there is no association, as discussed, for example, by Bush & Moore, Chapter 11: Genome-Wide Association Studies, PLOS Computational Biology 8(12):e1002822, 1-11 (2012). A genome-wide −log₁₀(p-value) corresponds to a p-value expressed on a logarithmic scale, for convenience, and corrected to take into account the effective number of statistical tests that have been carried out, based on multiple tests for association conducted with respect to an entire genome of a corresponding specific population, also as discussed by Bush & Moore (2012). Accordingly, a genome-wide −log₁₀(p-value) that is relatively high indicates that the likelihood that the observed test statistic, relating to association, would have been observed in the absence of association is extremely low.

Stratification and kinship correction are taken into account in determining the association. As noted above, stratification and kinship correction reduce false-positive signals due to recent common ancestry of small groups of individuals within the population of oil palm plants from which the test oil palm plant is sampled, thereby making practical the method for predicting palm oil yield of a test oil palm plant based on association.

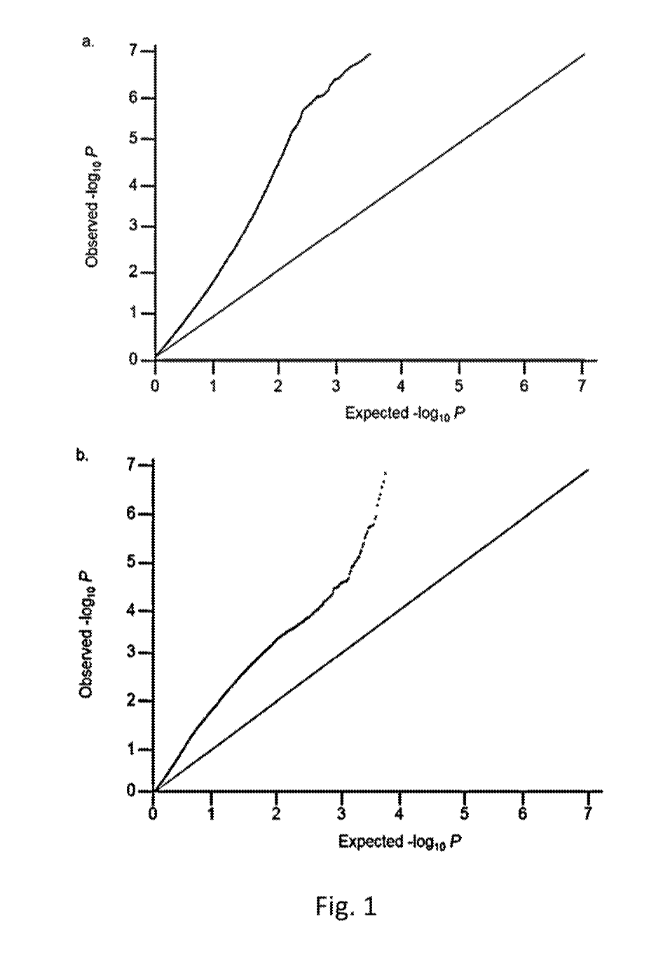

Of relevance here, a genome-wide association study (also termed GWAS) was performed on Deli x AVROS and Nigerian x AVROS, respectively using a naive model. The method only measured the association between the markers and the trait of interest regardless of population structures, or families, of the mapping population. According to quartile-quartile (Q-Q) plots and genomic inflation factor (GIF) estimations, −log₁₀(p-values) that were heavily inflated were observed, specifically indicating 4017 and 24760 SNPs to be associated with O/DM. As shown in FIG. 1, Deli x AVROS with GIP=3.66 and Nigerian x AVROS with GIF=11.9 indicated early deviation of the observed −log₁₀(p-value) from the null expectation (y=x), respectively. Most of these indicated SNPs only explained origin effects, not trait variants, and thus were false-positive signals. The naive model failed to account far the recent common ancestry of small groups of individuals, defined as cryptic relatedness, in accordance with Astle & Balding, Statistical Science 24:451-471 (2009), here posing a more serious confounding problem than population structure to the GWAS, in accordance with Devlin & Roeder, Biometrics 55:997-1004 (1999).

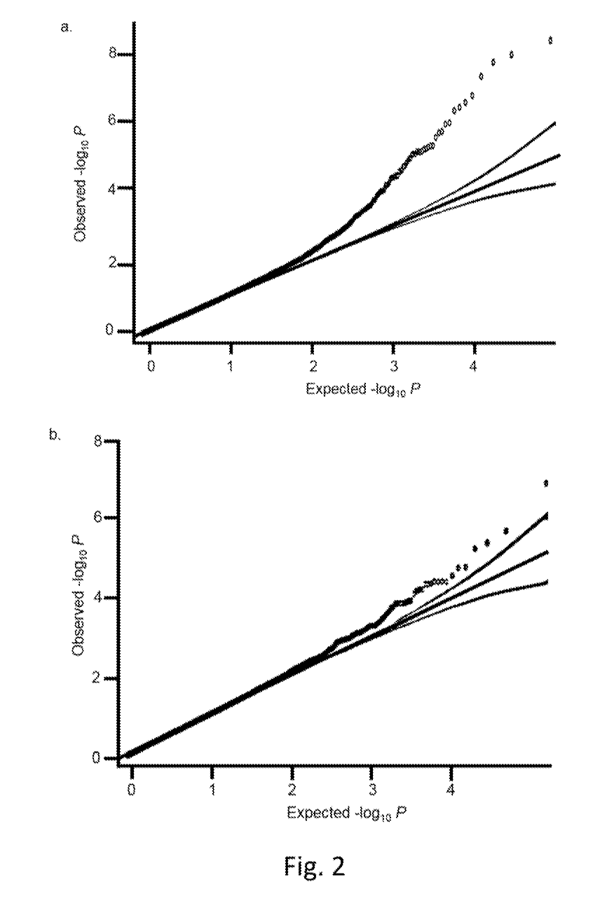

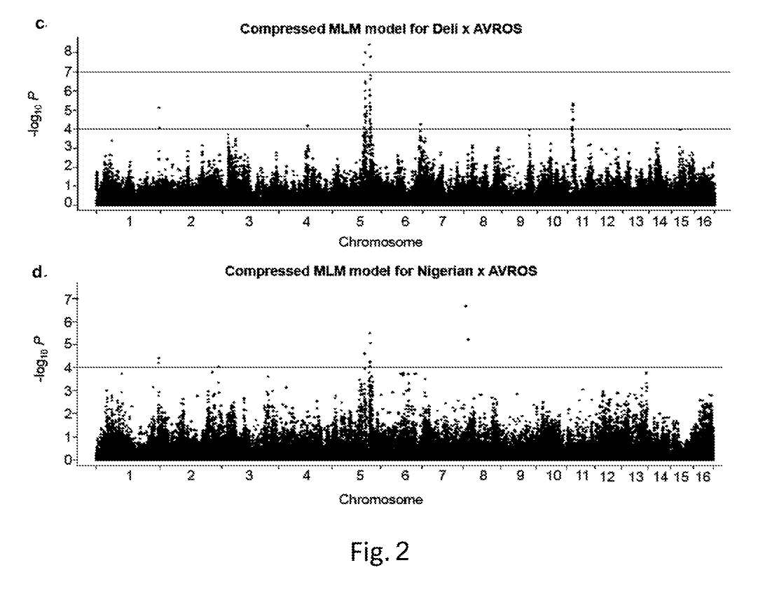

A subsequent GWAS based on a compressed mixed linear model (also termed MLM) with population parameters previously determined (P3D) was carried out toward addressing the problem of genomic inflations using principal component analysis and a group kinship matrix. This approach greatly reduced false positives, specifically resulting in 70 and 18 O/DM-associated SNPs in Deli x AVROS and Nigerian x AVROS, respectively. Specifically, as shown in FIG. 2, the Q-Q plots in both populations showed that deviation of the observed statistics from the null expectation were delayed significantly. Moreover, the GIFs for Deli x AVROS and Nigerian x AVROS also declined to 1.1 and 1.9 (approaching an ideal GIF=1.0), The chromosomal distribution of the resulting SNPs for both populations can be visualized in Manhattan plots, also shown in FIG. 2. Based on this approach, a total of 82 O/DM-associated SNPs were identified after excluding markers that overlapped in both populations.

Accordingly, for example, the first SNP marker being located in a first QTL for a high-oil-production trait and being associated, after stratification and kinship correction, with the high-oil-production trait with a genome-wide −log₁₀(p-value) of at least 4.0 in the population can be a SNP marker for which association with the high-oil-production trait (i) has been confirmed based on a model that is not a naive model and/or (ii) would be confirmed based on a model that is not a naive model. Also for example, the first SNP marker being located in a first QTL for a high-oil-production trait and being associated, after stratification and kinship correction, with the high-oil-production trait with a genome-wide −log₁₀(p-value) of at least 4.0 in the population can be a SNP marker for which association with the high-oil-production trait (i) has been confirmed based on a compressed mixed linear model with population parameters previously determined, carried out using principal component analysis and a group kinship matrix and/or (ii) would be confirmed based on a compressed mixed linear model with population parameters previously determined, carried out using principal component analysis and a group kinship matrix.

A first SNP marker having a linkage disequilibrium r² value of at least 0.2 with respect to a first other SNP marker that is linked thereto and associated, after stratification and kinship correction, with the high-oil-production trait with a genome-wide −log₁₀(p-value) of at least 4.0 in the population indicates the following. First, a high likelihood exists that an allele of the first SNP marker and an allele of the first other SNP marker are in linkage disequilibrium. Second, a high likelihood exists that the first other SNP marker and the trait are linked. In this regard, a linkage disequilibrium r² value relates to measuring likelihood that two loci are in linkage disequilibrium as an average pairwise correlation coefficient.

Accordingly, in some examples the first SNP marker is associated, after stratification and kinship correction, with the high-oil-production trait with a genome-wide −log₁₀(p-value) of at least 4.0 in the population. Also, in some examples the first SNP marker has a linkage disequilibrium r² value of at least 0.2 with respect to a first other SNP marker that is linked thereto and associated, after stratification and kinship correction, with the high-oil-production trait with a genome-wide −log₁₀(p-value) of at least 4.0 in the population. Also, in some examples both apply.

The first QTL can be a region of the oil palm genome corresponding to one of:
(1) QTL region 1, extending from nucleotide 66542323 to 66776312 of chromosome 1;
(2) QTL region 2, extending from nucleotide 66807385 to 67299617 of chromosome 1;
(3) QTL region 3, extending from nucleotide 62277032 to 62355782 of chromosome 2;
(4) QTL region 4, extending from nucleotide 31132787 to 3 173962 of chromosome 4;
(5) QTL region 5, extending from nucleotide 32863621 to 32964104 of chromosome 5;
(6) QTL region 6, extending from nucleotide 33355931 to 33509217 of chromosome 5;
(7) QTL region 7, extending from nucleotide 33658904 to 34233352 of chromosome 5;
(8) QTL region 8, extending from nucleotide 34358119 to 34997228 of chromosome 5;
(9) QTL region 9, extending from nucleotide: 35004388 to 35125743 of chromosome 5;
(10) QTL region 10, extending from nucleotide 35191678 to 35193677 of chromosome 5;
(11) QTL region 11, extending from nucleotide 36108847 to 36272808 of chromosome 5;
(12) QTL region 12, extending from nucleotide 39210662 to 39225076 of chromosome 5;
(13) QTL region 13, extending from nucleotide 39518005 to 40469897 of chromosome 5;
(14) QTL region 14, extending from nucleotide 40535309 to 40690150 of chromosome 5;
(15) QTL region 15, extending from nucleotide 40789706 to 40983955 of chromosome 5;
(16) QTL region 16, extending from nucleotide 41001085 to 41302446 of chromosome 5;
(17)QTL region 17, extending from nucleotide 3050807 to 3241977 of chromosome 8;
(18) QTL region 18, extending from nucleotide 5354764 to 5445890 of chromosome 8;
(19) QTL region 19, extending from nucleotide 29488933 to 29602300 of chromosome 9;
(20) QTL region 20, extending from nucleotide 4797284 to 5717606 of chromosome 11; or
(21) QTL region 21, extending from nucleotide 8611715 to 8857914 of chromosome 15.

The numbering of chromosomes, also termed linkage groups, and nucleotides thereof is in accordance with a 1.8 gigabase genuine sequence of the African oil palm E. guineensis as described by Singh et al., Nature 500:335-339 (2013) and the supplementary information noted therein, indicating that the E. guineensis BioProject is available for download at http://genomsawit.mpob.gov.my and has been registered at the NCBI under BioProject accession PRJNA192219 and that the Whole Genome Shotgun project has been deposited at DDBJ/EMBL/GenBank under the accession ASJS00000000.

For reference, QTL region 1 corresponds to the region of chromosome 1 of the genome of oil palm extending from the 5′ end of SEQ ID NO: 1 to the 3′ end of SEQ ID NO: 2. Similarly, QTL region 2 corresponds to the region of chromosome 1 extending from the 5′ end of SEQ ID NO: 3 to the 3′ end of SEQ ID NO: 4. QTL region 3 corresponds to the region of chromosome 2 extending from the 5′ end of SEQ ID NO: 5 to the 3′ end of SEQ ID NO: 6, QTL region 4 corresponds to the region of chromosome 4 extending from the 5′ end of SEQ ID NO: 7 to the 3′ end of SEQ ID NO: 8. QTL region 5 corresponds to the region of chromosome 5 extending from the 5′ end of SEQ ID NO: 9 to the 3′ end of SEQ ID NO: 10. QTL region 6 corresponds to the region of chromosome 5 extending from the 5′ end of SEQ ID NO: 11 to the 3′ end of SEQ ID NO: 12. QTL region 7 corresponds to the region of chromosome 5 extending from the 5′ end of SEQ ID NO: 13 to the 3′ end of SEQ ID NO: 14. QTL region 8 corresponds to the region of chromosome 5 extending from the 5′ end of SEQ ID NO: 15 to the 3′ end of SEQ ID NO: 16. QTL region 9 corresponds to the region of chromosome 5 extending from the 5′ end of SEQ ID NO: 17 to the 3′ end of SEQ ID NO: 18. QTL region 10 corresponds to the region of chromosome 5 extending from the 5′ end of SEQ ID NO: 19 to the 3′ end of SEQ ID NO: 20. QTL region 11 corresponds to the region of chromosome 5 extending from the 5′ end of SEQ ID NO: 21 to the 3′ end of SEQ ID NO: 22. QTL region 12 corresponds to the region of chromosome 5 extending from the 5′ end of SEQ ID NO: 23 to the 3′ end of SEQ ID NO: 24. QTL region 13 corresponds to the region of chromosome 5 extending from the 5′ end of SEQ ID NO: 25 to the 3′ end of SEQ ID NO: 26. QTL region 14 corresponds to the region of chromosome 5 extending from the 5′ end of SEQ ID NO: 27 to the 3′ end of SEQ ID NO: 28. QTL region 15 corresponds to the region of chromosome 5 extending from the 5′ end of SEQ ID NO: 29 to the 3′ end of SEQ ID NO: 30. QTL region 16 corresponds to the region of chromosome 5 extending front the 5′ end of SEQ ID NO: 31 to the 3′ end of SEQ ID NO: 32. QTL region 17 corresponds to the region of chromosome 8 extending from the 5′ end of SEQ ID NO: 33 to the 3′ end of SEQ ID NO: 34. QTL region 18 corresponds to the region of chromosome 8 extending front the 5′ end of SEQ ID NO: 35 to the 3′ end of SEQ ID NO: 36, QTL region 19 corresponds to the region of chromosome 9 extending from the 5′ end of SEQ ID NO: 37 to the 3′ end of SEQ ID NO: 38. QTL region 20 corresponds to the region of chromosome 11 extending from the 5′ end of SEQ ID NO: 39 to the 3′ end of SEQ ID NO: 40. QTL region 21 corresponds to the region of chromosome 15 extending from the 5′ end of SEQ ID NO: 41 to the 3′ end of SEQ ID NO: 42.

The method also comprises a step of (ii) comparing the first SNP genotype of the test oil palm plant to a corresponding first reference SNP genotype indicative of the high-oil-production trait in the same genetic background as the population. The genetic background that is the same as the population can correspond, for example, to a population based on crossing oil palm plants of the same types as used to generate the population from which the test oil palm plant is sampled, e.g. a Nigerian dura x AVROS pisifera population, a Deli dura x AVROS pisifera population, or a combination thereof, or a Nigerian dura x Nigerian dura population, a Nigerian dura x Deli dura population, a Deli dura x Deli dura population, an AVROS pisifira x AVROS tenera population, an AVROS tenera x AVROS genera population, or a combination thereof. The genetic background that is the same as the population also can correspond, for example, to a population based on crossing the same individual oil palm plants used to generate the population from which the test oil palm plant is sampled. The genetic background that is the same as the population also can correspond, for example, to the same actual population from which the test oil palm plant is sampled.

The first reference SNP genotype indicative of the high-oil-production trait in the same genetic background as the population can correspond to the same SNP as the first SNP genotype, i.e. both can correspond to the same polymorphic variation with respect to a single nucleotide that occurs at a particular locus of a particular chromosome. The first reference SNP genotype can comprise one or more SNP alleles that, alone or together, indicate a higher likelihood that the test oil palm plant thereof exhibits, if mature, or Will exhibit, upon reaching maturity, the high-oil-production trait, in comparison to oil palm plants of the same population that lack the one or more SNP alleles.

The method also comprises a step of (iii) predicting palm oil yield of the test oil palm plant based on the extent to which the first SNP genotype of the test oil palm plant matches the corresponding first reference SNP genotype. The first SNP genotype of the test oil palm plant can match the corresponding first reference SNP genotype based on both SNP genotypes sharing at least a first SNP allele indicative of the high-oil-production trait in the same genetic background as the population. In some examples the first SNP genotype and the first reference SNP genotype are heterozygous for the first allele indicative of the high-oil production trait, i.e. both have only one copy of the SNP allele. Also, in some examples the first SNP genotype and the first reference SNP genotype are homozygous for the first allele indicative of the high-oil production trait, i.e. both have two copies of the SNP allele. Also, in some examples the first SNP genotype is heterozygous for the first allele indicative of the high-oil production trait and the first reference SNP genotype is homozygous liar the first allele indicative of the high-oil production trait. Also, in some examples the first SNP genotype is homozygous for the first allele indicative of the high-oil production trait and the first reference SNP genotype is heterozygous for the first allele indicative of the high-oil production trait.

The step of predicting palm oil yield of the test oil palm plant can further comprise applying a model, such as a genotype model, a dominant model, or a recessive model, among others, in order to facilitate the predicting. A genotype model tests the association of a trait, e.g. a high-oil production trait, with the presence of a SNP allele, either a major allele (A) or a minor allele (a). A dominant model tests the association of a trait, e.g. a high-oil production trait, with the presence of a SNP allele either as a homozygous genotype or a heterozygous genotype, e.g. the major allele either as a homozygous genotype (e.g. A/A) or a heterozygous genotype (e.g. A/a). A recessive model tests the association of a trait, e.g. a high-oil production trait, with the presence of a SNP allele as a homozygous genotype, e.g. the major allele as a homozygous genotype (A/A). Accordingly, in some examples, the predicting of palm oil yield of the test oil palm plant further comprises applying a genotype model. Also in some examples, the predicting of palm oil yield of the test oil palm plant further comprises applying a dominant model. Also in some examples, the predicting of palm oil yield of the test oil palm plant further comprises applying a recessive model.

The degree to which a particular SNP genotype of a SNP marker in QTL regions 1 to 21 can be useful for predicting palm oil yield of a lest oil palm plant can depend on the source and breeding history of the breeding materials used to generate the population from which the test oil palm is sampled, including for example the extent to which one or more high-yield variant alleles that result in increases in palm oil yield have arisen within QTL regions 1 to 21 of the breeding materials and/or sources thereof used to generate the population, as well as the proximity of the one or more high-yield variant alleles to SNPs and the extent to which recombination has occurred between the SNPs and the high-yield variant alleles since the high-yield variant alleles arose. Factors such as proximity between a high-yield variant allele that promotes a high-oil-production trait and a SNP allele, a low number of generations since the high-yield variant allele arose, and a strong positive effect of the high-yield variant allele on palm oil production can tend to increase the degree to which of a particular SNP can be informative. These factors can vary, for example, depending on whether a high-yield variant allele is dominant or recessive, and thus whether a genotype model, a dominant model, or a recessive model may appropriately be applied with respect to a corresponding SNP allele. These factors also can vary, for example, between different populations generated by crosses of different individual palm plants.

The step of predicting palm oil yield of the test oil palm plant can be used advantageously not just to predict the palm oil yield of the test oil palm plant itself, but also to predict palm oil yields of progeny thereof. In this regard, oil palm breeders can use the method, as applied to a test oil palm plant that is a mother palm or a pollen donor, to determine possible SNP genotypes of progeny to be generated by crossing the test oil palm plant with another oil palm plant, and moreover can choose specific palms, i.e. the test oil palm plant and another specific oil palm plant that has been similarly characterized, to be crossed on this basis.

The method for predicting palm oil yield of a test oil palm plant can be used by focusing on particular QTLs, or combinations thereof, with respect to test oil palm plants derived from particular breeding materials.

For example, in some examples the population of oil palm plants comprises a Nigerian dura x AVROS pisifera population, the first QTL corresponds to one of QTL regions 2, 3, 8, 10, 13, 14, 16, 17, or 18, and step (iii) further comprises applying a genotype model, thereby predicting the palm a yield of the test oil palm plant.

Also, in some examples the population of oil palm plants comprises a Nigerian dura x AVROS pisifera population, the first QTL corresponds to one of QTL regions 3, 8, 10. 13, 15, 16, 17, or 18, and step (iii) further comprises applying a dominant model, thereby predicting the palm oil yield of the test oil palm plant.

Also in some examples the population of oil palm plants comprises a Nigerian dura x AVROS pisifera population, the first QTL corresponds to one of QTL regions 3, 4, 6, 7, 8, 9, 9, 11, 12, 13, 14, 16, 20, or 21, and step (iii) further comprises applying a recessive model, thereby predicting the palm oil yield of the test oil palm plant.

Also, in some examples the population of oil palm plants comprises a Deli dura x. AVROS pisifera population, the first QTL corresponds to one of QTL regions 1, 2, 4, 5, 6, 7, 8, 9, 11, 12, 13, 15, 16, 19, 20, or 21, and step (iii) further comprises applying a genotype model, thereby predicting the palm oil yield of the test oil palm plant.

Also, in some examples the population of oil palm plants comprises a Deli dura x AVROS pisifera population, the first QTL corresponds to one of QTL regions 8, 10, or 13, and step (iii) further comprises applying a dominant model, thereby predicting the palm oil yield of the test oil palm plant.

Also, in some examples the population of oil palm plants comprises a Deli dura x AVROS pisifera population, the first QTL corresponds to one of QTL regions 1, 2, 4, 5, 6, 7, 8, 9, 11, 12, 13, 15, 16, 19, 20, or 21, and step (iii) further comprises applying a recessive model, thereby predicting the palm oil yield of the test oil palm plant.

As noted above, crossing dura and pisifera gives rise to palms with a third fruit type, the tenera. As also noted, tenera are typically used as commercial planting materials. Accordingly, in some examples the test oil palm plant is a tenera candidate agricultural production plant. In some examples the population of oil palm plants comprises a Nigerian dura x AVROS pisifera population, and the test oil palm plant is a tenera candidate agricultural production plant. Also, in some examples the population of oil palm plants comprises a Deli dura x AVROS pisifera population, and the test oil palm plant is a tenera candidate agricultural production plant.

As also noted above, oil palm breeding is primarily aimed at selecting for improved parental dura and pisifera breeding stock palms for production of superior tenera commercial planting materials. As also noted, parental dura breeding populations are generated by crossing among selected dura palms, whereas pisifera palms are normally female sterile and thus breeding populations thereof must be generated by crossing among selected teneras or by crossing selected tenera with selected pisiferas. Accordingly, in some examples the test oil palm plant is a plant for mother palm selection and propagation, a plant for introgressed mother palm selection and propagation, or a plant for pollen donor selection and propagation. In some examples, the population of oil palm plants comprises a Nigerian dura x Nigerian dura population, and the test oil palm plant is a plant for mother palm selection and propagation. Also in some examples, the population of oil palm plants comprises a Nigerian dura x Nigerian dura population, and the test oil palm plant is a plant for introgressed mother palm selection and propagation. Also in some examples, the population of oil palm plants comprises a Deli dura x Deli dura population, and the test oil palm plant is a plant for mother palm selection and propagation. Also in some examples, the population of oil palm plants comprises an AVROS pisifera x AVROS tenera population, and the test oil palm plant is a plant for pollen donor selection and propagation. Also in some examples, the population of oil palm plants comprises an AVROS tenera x AVROS tenera population, and the test oil palm plant is a plant for pollen donor selection and propagation.

The method for predicting palm oil yield of a test oil palm plant also can be carried out by determining additional SNP genotypes, comparing the additional SNP genotypes to corresponding reference genotypes indicative of the high-oil-production trait, and further predicting palm oil yield of the test oil palm plant based on the extent to which the additional SNP genotypes match the corresponding reference SNP genotypes. This is because each SNP genotype can reflect a high-yield variant allele that contributes to a high-oil-production trait additively and/or synergistically with respect to the others.

Accordingly, in some examples step (i) further comprises determining, from the sample of the test oil palm plant, at least a second SNP genotype of the test oil palm plant, the second SNP genotype corresponding to a second SNP marker, the second SNP marker (a) being located in a second QTL For the high-oil-production trait and (b) being associated, after stratification and kinship correction, with the high-oil-production trait with a genome-wide −log ₁₀(p-value) of at least 4.0 in the population or having a linkage disequilibrium r² value of at least 0.2 with respect to a second other SNP marker that is linked thereto and associated, after stratification and kinship correction, with the high-oil-production trait with a genome-wide −log₁₀(p-value) of at least 4.0 in the population. Moreover, in these examples step (ii) further comprises comparing the second SNP genotype of the test oil palm plant to a corresponding second reference SNP genotype indicative of the high-oil-production trait in the same genetic background as the population. In addition, in these examples the second QTL corresponds to one or QTL, regions 1 to 21, with the proviso that the first. QTL and the second QTL correspond to different QTL regions. In some of these examples, step (iii) further comprises predicting palm oil yield of the test oil palm plant based on the extent to which the second SNP genotype of the test oil palm plant matches the corresponding second reference SNP genotype.

Also in some examples, step (i) further comprises determining, from the sample of the test oil palm plant, at least a third SNP genotype to a twenty-first SNP genotype of the test oil palm plant, the third SNP genotype to the twenty-first SNP genotype corresponding to a third SNP marker to a twenty-first SNP marker, respectively, the third SNP marker to the twenty-first SNP marker (a) being located in a third QTL to a twenty-first QTL, respectively, for the high-oil-production trait and (b) being associated, after stratification and kinship correction, with the high-oil-production trait with a genome-wide −log₁₀(p-value) of at least 4.0 in the population or having linkage disequilibrium r² values of at least 0.2 with respect to a third other SNP marker to a twenty-first other SNP marker, respectively, that are linked thereto and associated, after stratification and kinship correction, with the high-oil-production trait with a genome-wide −log₁₀(p-value) of at least 4.0 in the population. Moreover, in these examples step (ii) further comprises comparing the third SNP genotype to the twenty-first SNP genotype of the test oil palm plant to a corresponding third reference SNP genotype to a corresponding twenty-first reference SNP genotype, respectively, indicative of the high-oil-production trait in the same genetic background as the population. In addition, in these examples the third QTL to the twenty-first QTL each correspond to one of QTL regions 1 to 21, with the proviso that the first QTL to the twenty-first QTL each correspond to different QTL regions. In some of these examples, step (iii) further comprises predicting palm oil yield of the test oil palm plant based on the extent to which the third SNP genotype to the twenty-first SNP genotype of the test oil palm plant match the corresponding third reference SNP genotype to the corresponding twenty-first reference SNP genotype, respectively.

Also provided is a method of selecting a high-palm-oil-yielding oil palm plant for agricultural production of palm oil. The method comprises a step of (a) predicting palm oil yield of a test oil palm plant. This step can be carried out according to the method described above, i.e. including a step of (i) determining, from a sample of a test oil palm plant of a population of oil palm plants, at least a first single nucleotide polymorphism (SNP) genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first QTL is a region of the oil palm genome corresponding to one of QTL regions 1 to 21, as described above. The method also comprises a step of (b) field planting the test oil palm plant for agricultural production of palm oil if the palm oil yield of the test oil palm plant is predicted to be higher than average for the population based on step (a).

Also provided is a method of selecting a high-palm-oil-yielding oil palm plant for cultivation in cell culture. The method comprises a step of (a) predicting palm oil yield of a test oil palm plant. Again, this step can be carried out according to the method described above, i.e. including a step of (i) determining, from a sample of a test oil palm plant of a population of oil palm plants, at least a first single nucleotide polymorphism (SNP) genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first QTL is a region of the oil palm genome corresponding to one of QTL regions 1 to 21, as described above. The method also comprises a step of (b) subjecting at least one cell of the test oil palm plant to cultivation in cell culture if the palm oil yield of the test oil palm plant is predicted to be higher than average for the population based on step (a).

Also provided is a method of selecting a parental oil palm plant for use in breeding to obtain agricultural production plants or improved parental oil palm plants. As noted above, oil palm breeders can use the method, as applied to a test oil palm plant that is a mother palm or a pollen donor, to determine possible SNP genotypes of progeny to be generated by crossing the test oil palm plant with another oil palm plant, and moreover can choose specific palms, i.e. the test oil palm plant and another specific oil palm plant that has been similarly characterized, to be crossed on this basis. The method comprises a step of (a) predicting palm oil yield of a test oil palm plant. Again, this step can be carried out according to the method described above, i.e. including a step of (i) determining, from a sample of a test oil palm plant of a population of oil palm plants, at least a first single nucleotide polymorphism (SNP) genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first QTL is a region of the oil palm genome corresponding to one of QTL regions 1 to 21, as described above. The method also comprises a step of (b) selecting the test oil palm plant for use in breeding if the palm oil yield of tenera progeny of the test oil palm plant is predicted to be higher than average for the population based on step (a).

Also as noted above, a SNP detection kit for predicting palm oil yield of a test oil palm plant is disclosed. The kit comprises (i) a set of at least 21 nucleotide molecules suitable for determining, from a sample of a test oil palm plant of a population of oil palm plants, a first SNP genotype to a twenty-first SNP genotype, respectively, of the test oil palm plant. The first. SNP genotype to the twenty-first SNP genotype correspond to a first SNP marker to a twenty-first SNP marker, respectively. The first SNP marker to the twenty-first SNP marker are located in a first QTL to a twenty-first QTL, respectively, for a high-oil-production trait in the population. The first QTL to the twenty-first QTL are regions of the oil palm genome corresponding, respectively, to QTL regions 1 to 21, as described above. The first SNP marker to the twenty-first SNP marker also are associated, after stratification and kinship correction, with the high-oil-production trait with a genome-wide −log₁₀(p-value) of a least 4.0 in the population or have linkage disequilibrium r² values of at least 0.2 with respect to a first other SNP marker to a twenty-first other SNP marker, respectively, that are linked thereto and associated, after stratification and kinship correction, with the high-oil-production trait with a genomewide −log₁₀(p-value) of at least 4.0 in the population. The kit also comprises (ii) a reference sample of a reference high-oil-yielding oil palm plant of the population.

In some examples the SNP detection kit further comprises a solid substrate, the nucleotide molecules being attached to the solid substrate. Also in some examples, the nucleotide molecules am oligonucleotide or polynucleotides.

The following examples are for purposes of illustration and are not intended to limit the scope of the claims.

### EXAMPLE

### Sampling and DNA Preparation

For re-sequencing, 132 palms belonging to 59 origins maintained at Sime Darby Plantation R&D Centre in Malaysia were sampled. The sampling was extended to the genome-wide association study (also termed GWAS) mapping populations derived from Deli dura x AVROS pisifera breeding population (1,045 palms) and Nigerian dura x AVROS pisifera introgression line population (586 palms). The sample selection was based on a good representation of oil-to-dry mesocarp (also termed O/DM) variants and pedigree recorded by the corresponding breeders. Total genomic DNA was isolated from unopened spear leaves using the DNAeasy (R) Plant Mini Kit (Qiagen, Limburg, Netherlands).

### Whole-Genome Re-Sequencing

The 132 samples were pooled based on an equal molar concentration of DNA from each sample to form the sequencing DNA pool. A library was prepared for re-sequencing using HiSeq 2000 (TM) sequencing systems (Illumina, San Diego, Calif.) to generate 100-bp pair-end reads to a 35× genome coverage, resulting in 924,271,650 raw reads. The pair-end reads were trimmed, filtered, and aligned to the published oil palm genome, as described by Singh et al., Nature 500:335-339 (2013), using BWA Mapper, as published by Li & Durbin, Bioinformatics 26:589-595 (2010), with default parameters. A total of 7,755,949 putative SNPs were then called and filtered using SAMtools, as published by Li et al., Bioinformatics 25:2078-2079 (2009), with parameters of minimal mapping quality score of the SNP being 25, minimal depth 3×, and minimal SNP distance from a gap of 2 bp. Of the putative SNPs, 1,085,204 SNPs that were generated from Elaeis oleifera were removed. Also removed were 802,449 SNPs based on coverage (minimal 17 or maximal 53), genotype quality with minimal score of 8, and/or minor allele frequency (MAF<0.05). The other filtering steps were performed to remove 5,274,408 SNPs based on technical requirement of Illumina, including removal of pairs of SNPs with distance less than 60 bp and ambiguous nucleotides. This yielded 593,888 quality SNPs. According to linkage disequilibrium, r² cutoff being set at 0.3, a total of 100,000 SNPs with an average density of one SNP per 16 Kb were submitted to Illumine for design score calculation using Illumina's Assay Design Tool for Infinium (Illumina).

### SNP Genotyping

An OP100K Infinium array (Illumina) was used to assay the GWAS mapping populations (˜250 ng DNA/sample). The overnight amplified DNA samples were then fragmented by a controlled enzymatic process that did not require gel electrophoresis. The re-suspended DNA samples were hybridized to BeadChips (Illumina) after an overnight incubation in a corresponding capillary flow-through chamber. Allele specific hybridizations were fluorescently labeled and detected by a BeadArray Reader (Illumina). The raw reads were then analyzed using GenomeStudio Data Analysis software (Illumina) for automated genotyping calling and quality control. To generate the genotypic dataset for GWAS, only the SNPs that had minor allele frequency (also termed MAF)>0.01 and >90% of call rate were accepted. The missing genotype of those SNPs was subsequently imputed based on the mean of each marker, in accordance with Endelman, Plant Genome 4:250-255 (2011).

### Genetic Stratification and Population Analyses

Neighbor-joining (also termed NJ) tree was used to infer the genetic stratification of the GWAS mapping populations. A Hamming's pairwise distance matrix for all SNP sites was calculated to plot the NJ tree. The genome-wide linkage disequilibrium (also termed LD) decay rates in the Deli x AVROS and Nigerian x AVROS were important to anticipate the requirements for suitable mapping resolution of the SNP for GWAS. The rate is defined as the chromosomal distance at which the average pairwise correlation coefficient (r²) dropped to the half of its maximum value. In this study, pairwise r² for all SNPs in a 1-Kb window were calculated and averaged across the whole genome based on composite method in the R package SNPrelate, in accordance with Zheng et al., Bioinformatics 28:3326-3328 (2012).

### Phenotypic Data Compilation and GWAS

O/DM is a direct measurement of crude palm oil (CPO) extracted from dry mesocarp tissue using a solvent. To measure O/DM, approximately 30 g of fertile fruits were randomly sampled per bunch from a minimum of three bunches per palm (≥4 years after field planting of the palms), resulting in a reliable mean O/DM. The O/DM difference between the Deli x AVROS and Nigerian x AVROS populations were tested for significance by a Student-t test. Subsequently, association analyses were conducted on 1,459 Deli x AVROS and 586 Nigerian x AVROS, respectively, based on a naive model in an R package GenABEL, in accordance with Aulchenko et al., Bioinformatics 23:1294-1296 (2007), and the compressed mixed linear model (also termed MLM) with P3D analysis according to Zhang et al., Nature Genetics 42:355-360 (2010), in the rrBLUP program, in accordance with Endelman (2011). The total. number of common SNPs was 55,054 SNPs with MAF≥0.01. Genetic sub-structure resulting from cryptic relatedness was accounted for by including kinship matrix, in accordance with VanRaden, Journal of Dairy Science 91:4414-4423 (2008), as a random effect in the compressed MLM method. The whole-genome significance −log₁₀(p-value) cutoff was fixed at ≥4.0 and ≥7.0, based on a Bonferroni correction method. The quartile-quartile (Q-Q) plots and Manhattan plots were then constructed using yin R package qqman, in accordance with Turner, qqman An R package for visualizing GWAS results using Q-Q and Manhattan plots, available at http://biorxiv.org/content/early/2014/05/14/005165 (last accessed Nov. 15, 2014). Inflated false-positive signals were also evaluated for both methods according to the genomic inflated factor (GIF) estimated in an R package GenABEL, in accordance with Aulchenko et al. (2007).

### SNP Effects and Statistical Analyses

The significant SNPs according to −log₁₀(p-value) ≥4.0 were further analyzed for the genotype model-based SNP effects on O/DM trait, illustrated in boxplots and followed by one-way ANOVA test with multi comparisons using Minitab 14, in accordance with Du Feu et al., MINITAB 14, Teaching Statistics 27: 30-32 (2005). The same analytical method was expanded to determine O/DM association with the presence of one SNP allele, either a major allele (A) or a minor allele (a) through dominance model (A/A+A/a. a/a) and recessive model (A/A, A/a+a/a).

### Results

O/DM phenotype data for the Deli x AVROS population and the Nigerian x AVROS population, expressed as percentage O/DM are provided in TABLE 1. As can be seen, the Nigerian x AVROS population exhibited a mean percentage O/DM of 75.67%, and the Deli x AVROS population exhibited a mean percentage O/DM of 76.87%.

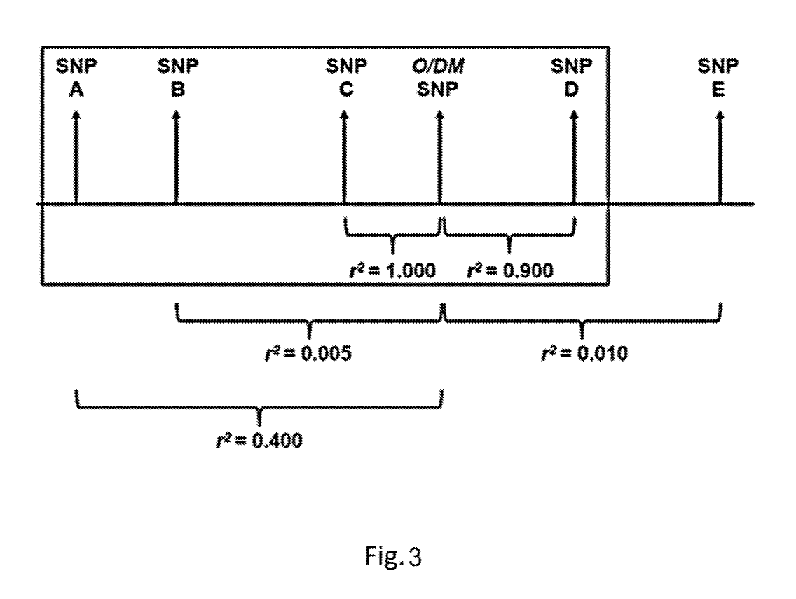

Twenty-one QTL regions for O/DM phenotypes in the Nigerian x AVROS population and the Deli x AVROS population were identified, as shown in TABLE 2, with elaboration in FIG. 3. The numbering of chromosomes and nucleotides thereof is in accordance with the 1.8 gigabase genome sequence of the African oil palm E. guineensis as described by Singh et al., Nature 500:335-339 (2013) and the supplementary information noted therein, as discussed above. The 21 QTL regions span 5,779,750 nucleotides, corresponding to approximately 0.3% of the oil palm genome.

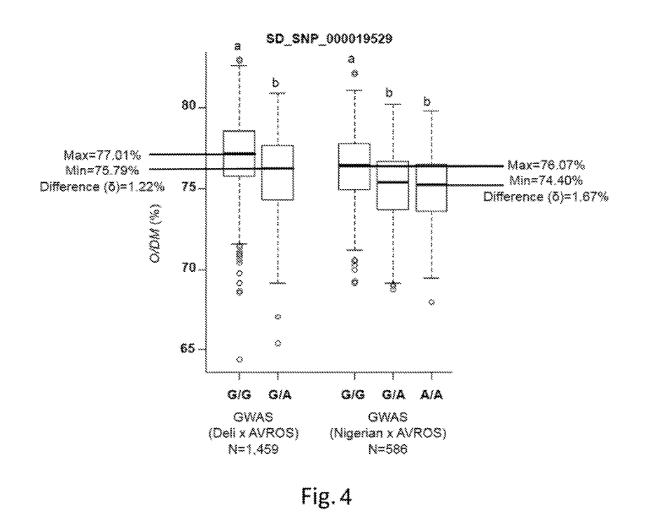

Eighty-two SNP markers that are informative with respect to O/DM for the Nigerian x AVROS population and/or the Deli x AVROS population and that are located within the 21 QTLs were identified, as shown in TABLE 3, TABLE 4, TABLE 5, TABLE 6, and FIG. 4. SNP identifying information and positional information is provided in TABLE 3, As can be seen in TABLE 4 and TABLE 5, each of the SNP markers yielded a genome-wide −log₁₀(p-value) of at least 4.0 in at least one of the Nigerian x AVROS population and/or the Deli x AVROS population with respect to at least one of a genotype model, a dominant model, or a recessive model. Indeed, many of the SNP markers yielded a fenome-wide −log₁₀(p-value) of at least 4.0 in both populations and/or with respect to more than one of the models. Also, as can be seen in TABLE 6, for each of the SNP markers for which a minor SNP allele was detected in a given population, differences (termed δ) in mean percentage O/DM for oil palm plants of the given population including a SNP allele associated with the high-oil-production trait (termed Max) versus oil palm plants of the given population lacking the SNP allele (termed Min), with respect to the genotype model in particular, ranged from 0.14% to 4.09% for the Nigerian x AVROS population and range from 0.32% to 7.40% for the Deli x AVROS population. As shown in more detail in FIG. 4, various SNP markers are informative with respect to both populations.

TABLE 1
Oil-to-dry mesocarp, expressed as percentages, for the Deli × AVROS
population and the Nigerian × AVROS population.

| | Mean | | | Median | |
|---|---|---|---|---|---|
| Population | (%) | St. Dev. | Coef. Var. | (%) | Range (%) |
| Nigerian × AVROS | 75.67% | 2.43 | 3.22 | 75.80% | 14.20% |
| Deli × AVROS | 76.87% | 2.28 | 2.96 | 77.10% | 18.60% |

TABLE 2
QTL regions 1 to 21: Chromosome and nucleotide position information.

| | | Start | Stop | Length |
|---|---|---|---|---|
| QTL region | Chromosome | nucleotide | nucleotide | (Stop-Start + 1) |
| 1 | 1 | 66542323 | 66776312 | 233990 |
| 2 | 1 | 66807385 | 67299617 | 492233 |
| 3 | 2 | 62277032 | 62355782 | 78751 |
| 4 | 4 | 31132787 | 31173962 | 41176 |
| 5 | 5 | 32863621 | 32964104 | 100484 |
| 6 | 5 | 33355931 | 33509217 | 153287 |
| 7 | 5 | 33658904 | 34233352 | 574449 |
| 8 | 5 | 34358119 | 34997228 | 639110 |
| 9 | 5 | 35004388 | 35125743 | 121356 |
| 10 | 5 | 35191678 | 35193678 | 2001 |
| 11 | 5 | 36108847 | 36272808 | 163962 |
| 12 | 5 | 39210662 | 39225076 | 14415 |
| 13 | 5 | 39518005 | 40469897 | 951893 |
| 14 | 5 | 40535309 | 40690150 | 154842 |
| 15 | 5 | 40789706 | 40983955 | 194250 |
| 16 | 5 | 41001085 | 41302446 | 301362 |
| 17 | 8 | 3050807 | 3241977 | 191171 |
| 18 | 8 | 5354764 | 5445890 | 91127 |
| 19 | 9 | 29488933 | 29602300 | 113368 |
| 20 | 11 | 4797284 | 5717606 | 920323 |
| 21 | 15 | 8611715 | 8857914 | 246200 |

TABLE 3
SNP markers in QTL regions 1 to 21: SNP identifying information
and positional information.

| SNP | | | | |
|---|---|---|---|---|
| No. | SNP ID | QTL region | Chromosome | Position |
| 1 | SD_SNP_000002127 | 1 | 1 | 66639699 |
| 2 | SD_SNP_000016244 | 2 | 1 | 66972538 |
| 3 | SD_SNP_000013063 | 2 | 1 | 67033874 |
| 4 | SD_SNP_000049433 | 2 | 1 | 67248054 |
| 5 | SD_SNP_000038645 | 3 | 2 | 62287970 |
| 6 | SD_SNP_000006192 | 4 | 4 | 31149521 |
| 7 | SD_SNP_000049049 | 5 | 5 | 32866989 |
| 8 | SD_SNP_000039298 | 6 | 5 | 33457617 |
| 9 | SD_SNP_000016161 | 7 | 5 | 33975929 |
| 10 | SD_SNP_000016164 | 7 | 5 | 34027909 |
| 11 | SD_SNP_000013028 | 7 | 5 | 34142044 |
| 12 | SD_SNP_000003832 | 8 | 5 | 34454937 |
| 13 | SD_SNP_000018373 | 8 | 5 | 34521370 |
| 14 | SD_SNP_000018372 | 8 | 5 | 34525454 |
| 15 | SD_SNP_000037414 | 8 | 5 | 34568717 |
| 16 | SD_SNP_000037422 | 8 | 5 | 34603394 |
| 17 | SD_SNP_000040073 | 8 | 5 | 34612126 |
| 18 | SD_SNP_000022444 | 8 | 5 | 34773282 |
| 19 | SD_SNP_000010418 | 8 | 5 | 34828628 |
| 20 | SD_SNP_000015218 | 8 | 5 | 34856258 |
| 21 | SD_SNP_000015219 | 8 | 5 | 34863191 |
| 22 | SD_SNP_000042931 | 8 | 5 | 34980654 |
| 23 | SD_SNP_000041945 | 9 | 5 | 35070248 |
| 24 | SD_SNP_000048207 | 9 | 5 | 35080572 |
| 25 | SD_SNP_000024668 | 9 | 5 | 35100527 |
| 26 | SD_SNP_000024664 | 9 | 5 | 35121695 |
| 27 | SD_SNP_000050827 | 10 | 5 | 35192678 |
| 28 | SD_SNP_000033957 | 11 | 5 | 36158880 |
| 29 | SD_SNP_000030440 | 11 | 5 | 36218554 |
| 30 | SD_SNP_000030409 | 11 | 5 | 36234729 |
| 31 | SD_SNP_000024845 | 12 | 5 | 39210662 |
| 32 | SD_SNP_000054111 | 13 | 5 | 39607208 |
| 33 | SD_SNP_000054110 | 13 | 5 | 39610847 |
| 34 | SD_SNP_000054109 | 13 | 5 | 39613906 |
| 35 | SD_SNP_000054992 | 13 | 5 | 39620126 |
| 36 | SD_SNP_000054080 | 13 | 5 | 39642505 |
| 37 | SD_SNP_000053315 | 13 | 5 | 39653455 |
| 38 | SD_SNP_000051833 | 13 | 5 | 39763460 |
| 39 | SD_SNP_000047120 | 13 | 5 | 39799450 |
| 40 | SD_SNP_000047117 | 13 | 5 | 39804720 |
| 41 | SD_SNP_000046882 | 13 | 5 | 39805514 |
| 42 | SD_SNP_000047116 | 13 | 5 | 39806797 |
| 43 | SD_SNP_000048815 | 13 | 5 | 39860983 |
| 44 | SD_SNP_000014128 | 13 | 5 | 39966907 |
| 45 | SD_SNP_000019028 | 13 | 5 | 40066844 |
| 46 | SD_SNP_000022774 | 13 | 5 | 40112108 |
| 47 | SD_SNP_000022773 | 13 | 5 | 40129189 |
| 48 | SD_SNP_000022770 | 13 | 5 | 40145585 |
| 49 | SD_SNP_000022766 | 13 | 5 | 40158838 |
| 50 | SD_SNP_000026602 | 13 | 5 | 40249343 |
| 51 | SD_SNP_000026599 | 13 | 5 | 40269392 |
| 52 | SD_SNP_000019529 | 13 | 5 | 40300709 |
| 53 | SD_SNP_000002370 | 13 | 5 | 40396733 |
| 54 | SD_SNP_000002372 | 13 | 5 | 40405137 |
| 55 | SD_SNP_000016503 | 14 | 5 | 40577880 |
| 56 | SD_SNP_000030214 | 14 | 5 | 40587726 |
| 57 | SD_SNP_000030215 | 14 | 5 | 40597291 |
| 58 | SD_SNP_000020190 | 15 | 5 | 40902353 |
| 59 | SD_SNP_000020192 | 15 | 5 | 40916454 |
| 60 | SD_SNP_000005964 | 16 | 5 | 41036282 |
| 61 | SD_SNP_000022588 | 16 | 5 | 41189141 |
| 62 | SD_SNP_000009135 | 16 | 5 | 41200140 |
| 63 | SD_SNP_000009134 | 16 | 5 | 41203008 |
| 64 | SD_SNP_000009133 | 16 | 5 | 41204629 |
| 65 | SD_SNP_000038512 | 17 | 8 | 3154572 |
| 66 | SD_SNP_000021743 | 18 | 8 | 5393101 |
| 67 | SD_SNP_000002970 | 19 | 9 | 29537541 |
| 68 | SD_SNP_000043748 | 20 | 11 | 4828172 |
| 69 | SD_SNP_000043747 | 20 | 11 | 4831923 |
| 70 | SD_SNP_000043745 | 20 | 11 | 4838915 |
| 71 | SD_SNP_000047737 | 20 | 11 | 5165520 |
| 72 | SD_SNP_000037573 | 20 | 11 | 5204949 |
| 73 | SD_SNP_000053510 | 20 | 11 | 5255710 |
| 74 | SD_SNP_000031828 | 20 | 11 | 5369351 |
| 75 | SD_SNP_000031829 | 20 | 11 | 5372237 |
| 76 | SD_SNP_000046132 | 20 | 11 | 5412986 |
| 77 | SD_SNP_000002502 | 20 | 11 | 5420279 |
| 78 | SD_SNP_000002504 | 20 | 11 | 5422706 |
| 79 | SD_SNP_000002507 | 20 | 11 | 5436885 |
| 80 | SD_SNP_000002508 | 20 | 11 | 5439423 |
| 81 | SD_SNP_000002510 | 20 | 11 | 5442401 |
| 82 | SD_SNP_000015708 | 21 | 15 | 8740489 |

TABLE 4
SNP markers in QTL regions 1 to 21; Nigerian x AVROS population
major allele, minor allele, minor allele frequency, and genome-wide
−log₁₀(p-value) with respect to a genotype model, a dominant model,
and a recessive model. SNP numbering is in accordance with Table 3.

| | Nigerian x AVROS | | | | | |
|---|---|---|---|---|---|---|
| | | | Minor | | | |
| SNP | Major | Minor | allele | [−log₁₀ (p-value)] | | |
| No. | allele | allele | frequency | Genotype | Dominant | Recessive |
| 1 | A | G | 0.352 | 0.397 | 1.059 | 0.222 |
| 2 | A | G | 0.386 | 4.197 | 1.034 | 0.894 |
| 3 | A | G | 0.307 | 0.937 | 0.875 | 3.720 |
| 4 | A | C | 0.234 | 4.405 | 3.238 | 0.736 |
| 5 | G | A | 0.123 | 4.023 | 4.517 | 9.783 |
| 6 | G | A | 0.112 | 0.267 | 0.000 | 4.864 |
| 7 | A | G | 0.242 | 0.030 | 3.014 | 2.926 |
| 8 | G | A | 0.354 | 0.151 | 2.104 | 4.893 |
| 9 | G | A | 0.292 | 0.752 | 0.827 | 1.671 |
| 10 | A | G | 0.392 | 0.185 | 2.065 | 6.754 |
| 11 | C | A | 0.089 | 1.117 | 2.356 | 1.410 |
| 12 | G | A | 0.487 | 0.585 | 0.743 | 0.827 |
| 13 | G | A | 0.320 | 0.862 | 0.037 | 3.146 |
| 14 | G | A | 0.422 | 0.577 | 1.723 | 0.294 |
| 15 | G | A | 0.300 | 0.491 | 0.265 | 10.991 |
| 16 | G | A | 0.384 | 2.159 | 2.334 | 0.322 |
| 17 | G | A | 0.176 | 3.062 | 0.000 | 4.431 |
| 18 | A | G | 0.389 | 0.106 | 0.000 | 0.552 |
| 19 | G | A | 0.247 | 4.610 | 8.656 | 19.818 |
| 20 | C | A | 0.327 | 2.894 | 7.776 | 8.549 |
| 21 | A | G | 0.300 | 2.381 | 1.951 | 13.930 |
| 22 | A | G | 0.356 | 3.943 | 0.000 | 11.737 |
| 23 | A | C | 0.221 | 0.183 | 0.724 | 1.835 |
| 24 | G | A | 0.196 | 0.142 | 0.994 | 4.062 |
| 25 | A | G | 0.320 | 1.340 | 0.320 | 0.484 |
| 26 | A | G | 0.298 | 2.577 | 2.019 | 14.305 |
| 27 | A | G | 0.248 | 4.595 | 9.229 | 19.216 |
| 28 | G | A | 0.445 | 0.346 | 0.117 | 1.191 |
| 29 | A | G | 0.333 | 0.855 | 0.000 | 7.883 |
| 30 | G | A | 0.394 | 0.787 | 0.095 | 5.970 |
| 31 | A | G | 0.166 | 2.858 | 0.000 | 4.263 |
| 32 | A | G | 0.379 | 0.179 | 1.931 | 1.240 |
| 33 | G | A | 0.379 | 0.179 | 1.931 | 1.240 |
| 34 | G | A | 0.478 | 0.242 | 4.299 | 0.607 |
| 35 | A | G | 0.478 | 0.242 | 4.299 | 0.607 |
| 36 | C | A | 0.378 | 0.191 | 1.733 | 1.347 |
| 37 | G | A | 0.444 | 1.851 | 9.576 | 12.882 |
| 38 | A | C | 0.491 | 3.786 | 3.271 | 11.613 |
| 39 | A | G | 0.489 | 2.107 | 1.875 | 3.854 |
| 40 | G | A | 0.489 | 2.107 | 1.875 | 3.854 |
| 41 | G | A | 0.489 | 2.107 | 1.875 | 3.854 |
| 42 | A | G | 0.417 | 1.680 | 0.234 | 2.666 |
| 43 | G | A | 0.451 | 3.450 | 8.040 | 0.293 |
| 44 | A | G | 0.340 | 2.598 | 0.000 | 5.191 |
| 45 | G | A | 0.493 | 3.013 | 0.069 | 7.855 |
| 46 | G | A | 0.350 | 1.974 | 11.362 | 7.961 |
| 47 | C | A | 0.308 | 2.402 | 2.830 | 9.053 |
| 48 | A | G | 0.409 | 4.042 | 9.662 | 1.574 |
| 49 | A | G | 0.358 | 5.499 | 0.000 | 12.136 |
| 50 | G | A | 0.312 | 0.186 | 1.720 | 5.460 |
| 51 | G | A | 0.350 | 3.547 | 1.526 | 0.130 |
| 52 | A | G | 0.323 | 4.213 | 2.198 | 9.978 |
| 53 | A | C | 0.200 | 4.194 | 1.768 | 12.208 |
| 54 | A | G | 0.456 | 0.387 | 2.306 | 6.013 |
| 55 | G | A | 0.223 | 4.057 | 1.997 | 8.377 |
| 56 | A | G | 0.223 | 4.057 | 1.997 | 8.377 |
| 57 | A | G | 0.217 | 4.259 | 1.656 | 8.969 |
| 58 | A | G | 0.397 | 2.776 | 4.930 | 0.648 |
| 59 | G | A | 0.419 | 2.564 | 2.685 | 0.127 |
| 60 | A | G | 0.322 | 5.058 | 10.310 | 17.361 |
| 61 | A | G | 0.207 | 0.592 | 0.609 | 6.294 |
| 62 | G | A | 0.148 | 4.259 | 0.000 | 10.480 |
| 63 | A | G | 0.148 | 4.259 | 0.000 | 10.480 |
| 64 | G | A | 0.148 | 4.259 | 0.000 | 10.480 |
| 65 | G | A | 0.095 | 6.669 | 10.173 | 0.950 |
| 66 | C | A | 0.109 | 5.213 | 12.135 | 1.206 |
| 67 | 0 | G | 0.000 | 0.000 | 0.000 | 0.000 |
| 68 | A | G | 0.041 | 0.000 | 0.000 | 8.885 |
| 69 | G | A | 0.176 | 0.332 | 0.000 | 3.777 |
| 70 | G | A | 0.176 | 0.332 | 0.000 | 3.777 |
| 71 | 0 | G | 0.000 | 0.000 | 0.000 | 0.000 |
| 72 | 0 | G | 0.000 | 0.000 | 0.000 | 0.000 |
| 73 | C | A | 0.259 | 0.190 | 0.000 | 0.708 |
| 74 | A | C | 0.390 | 0.054 | 0.000 | 1.460 |
| 75 | G | A | 0.390 | 0.054 | 0.000 | 1.460 |
| 76 | A | G | 0.049 | 0.000 | 0.000 | 8.090 |
| 77 | A | G | 0.049 | 0.000 | 0.000 | 8.090 |
| 78 | G | A | 0.051 | 1.089 | 0.000 | 9.112 |
| 79 | G | A | 0.049 | 0.000 | 0.000 | 8.090 |
| 80 | G | A | 0.213 | 0.282 | 0.000 | 3.444 |
| 81 | C | A | 0.049 | 0.000 | 0.000 | 8.090 |
| 82 | A | G | 0.184 | 0.077 | 0.000 | 16.667 |

TABLE 5
SNP markers in QTL regions 1 to 21: Deli x AVROS population
major allele, minor allele, minor allele frequency, and genome-wide
−log₁₀(p-value) with respect to a genotype model, a dominant model,
and a recessive model. SNP numbering is in accordance with Table 3.

| | Deli x AVROS | | | | | |
|---|---|---|---|---|---|---|
| | | | Minor | | | |
| SNP | Major | Minor | allele | [−log₁₀ (p-value)] | | |
| No. | allele | allele | frequency | Genotype | Dominant | Recessive |
| 1 | A | G | 0.061 | 4.979 | 0.000 | 7.86 |
| 2 | G | A | 0.459 | 0.149 | 0.352 | 0.08 |
| 3 | A | G | 0.066 | 4.014 | 0.000 | 7.22 |
| 4 | A | C | 0.131 | 0.201 | 0.000 | 1.20 |
| 5 | G | A | 0.001 | 0.000 | 0.000 | 0.80 |
| 6 | G | A | 0.126 | 4.144 | 0.000 | 5.20 |
| 7 | A | G | 0.072 | 7.532 | 0.000 | 10.41 |
| 8 | A | G | 0.383 | 4.467 | 1.012 | 5.19 |
| 9 | G | A | 0.083 | 5.230 | 0.000 | 8.93 |
| 10 | G | A | 0.378 | 4.155 | 0.741 | 5.23 |
| 11 | C | A | 0.147 | 4.081 | 2.238 | 6.01 |
| 12 | G | A | 0.389 | 4.692 | 0.741 | 6.37 |
| 13 | G | A | 0.142 | 4.721 | 1.930 | 8.00 |
| 14 | G | A | 0.154 | 6.312 | 2.937 | 9.55 |
| 15 | G | A | 0.407 | 4.090 | 3.322 | 6.34 |
| 16 | G | A | 0.376 | 5.562 | 3.277 | 8.30 |
| 17 | G | A | 0.077 | 6.882 | 0.000 | 10.97 |
| 18 | A | G | 0.151 | 4.793 | 0.000 | 9.36 |
| 19 | G | A | 0.383 | 5.957 | 3.501 | 8.00 |
| 20 | C | A | 0.169 | 8.555 | 4.315 | 8.60 |
| 21 | A | G | 0.385 | 4.958 | 3.551 | 8.23 |
| 22 | G | A | 0.386 | 5.546 | 3.961 | 8.54 |
| 23 | C | A | 0.302 | 4.250 | 3.026 | 7.59 |
| 24 | A | G | 0.298 | 4.712 | 3.026 | 7.73 |
| 25 | A | G | 0.079 | 6.748 | 0.000 | 13.64 |
| 26 | A | G | 0.095 | 5.021 | 0.000 | 8.40 |
| 27 | A | G | 0.315 | 0.819 | 4.252 | 0.26 |
| 28 | G | A | 0.072 | 4.819 | 0.063 | 7.93 |
| 29 | A | G | 0.077 | 4.509 | 0.000 | 6.93 |
| 30 | G | A | 0.080 | 4.622 | 0.001 | 7.33 |
| 31 | A | G | 0.070 | 8.630 | 0.000 | 11.94 |
| 32 | G | A | 0.303 | 5.571 | 3.808 | 8.71 |
| 33 | A | G | 0.302 | 5.154 | 3.524 | 8.71 |
| 34 | A | G | 0.296 | 5.731 | 3.524 | 10.08 |
| 35 | G | A | 0.296 | 5.731 | 3.524 | 10.08 |
| 36 | A | C | 0.303 | 5.570 | 3.808 | 8.71 |
| 37 | G | A | 0.388 | 6.202 | 4.283 | 8.27 |
| 38 | C | A | 0.382 | 6.062 | 3.927 | 8.84 |
| 39 | G | A | 0.427 | 4.636 | 1.973 | 1.18 |
| 40 | A | G | 0.426 | 4.510 | 1.973 | 1.12 |
| 41 | A | G | 0.426 | 4.638 | 1.973 | 1.12 |
| 42 | G | A | 0.426 | 4.618 | 1.973 | 1.12 |
| 43 | G | A | 0.375 | 6.270 | 3.402 | 9.56 |
| 44 | A | G | 0.132 | 5.764 | 0.000 | 4.15 |
| 45 | A | G | 0.181 | 7.083 | 3.873 | 8.53 |
| 46 | G | A | 0.416 | 4.509 | 7.385 | 0.33 |
| 47 | C | A | 0.415 | 4.737 | 7.451 | 0.34 |
| 48 | A | G | 0.372 | 4.153 | 3.050 | 8.19 |
| 49 | A | G | 0.381 | 4.242 | 0.000 | 5.52 |
| 50 | A | G | 0.287 | 4.249 | 3.331 | 9.11 |
| 51 | G | A | 0.077 | 8.378 | 0.000 | 11.68 |
| 52 | A | G | 0.078 | 7.365 | 0.000 | 11.23 |
| 53 | A | C | 0.081 | 5.998 | 0.000 | 9.55 |
| 54 | G | A | 0.318 | 4.002 | 3.186 | 9.89 |
| 55 | G | A | 0.265 | 0.857 | 0.551 | 3.72 |
| 56 | A | G | 0.263 | 0.856 | 0.588 | 3.51 |
| 57 | A | G | 0.271 | 0.546 | 0.493 | 3.32 |
| 58 | A | G | 0.073 | 5.188 | 0.000 | 10.75 |
| 59 | G | A | 0.076 | 4.935 | 0.000 | 10.75 |
| 60 | A | G | 0.073 | 4.406 | 0.000 | 10.18 |
| 61 | G | A | 0.404 | 4.192 | 0.000 | 7.71 |
| 62 | A | G | 0.398 | 3.191 | 0.000 | 6.89 |
| 63 | A | G | 0.080 | 2.814 | 0.000 | 8.66 |
| 64 | G | A | 0.080 | 2.813 | 0.000 | 8.68 |
| 65 | G | A | 0.266 | 0.634 | 0.671 | 0.60 |
| 66 | C | A | 0.299 | 0.338 | 1.889 | 1.91 |
| 67 | A | G | 0.242 | 4.052 | 0.928 | 5.93 |
| 68 | A | G | 0.251 | 4.258 | 2.231 | 6.11 |
| 69 | G | A | 0.265 | 4.197 | 1.512 | 6.75 |
| 70 | G | A | 0.265 | 4.221 | 1.512 | 6.74 |
| 71 | A | G | 0.250 | 5.091 | 2.186 | 7.63 |
| 72 | A | G | 0.241 | 4.565 | 0.674 | 7.41 |
| 73 | C | A | 0.279 | 4.097 | 1.651 | 6.92 |
| 74 | A | C | 0.259 | 4.945 | 3.200 | 6.73 |
| 75 | G | A | 0.281 | 5.292 | 3.571 | 7.63 |
| 76 | A | G | 0.257 | 4.548 | 1.886 | 5.38 |
| 77 | A | G | 0.251 | 5.311 | 2.293 | 6.63 |
| 78 | G | A | 0.257 | 4.481 | 1.886 | 5.38 |
| 79 | G | A | 0.251 | 5.242 | 2.293 | 6.63 |
| 80 | G | A | 0.257 | 4.486 | 1.886 | 5.38 |
| 81 | C | A | 0.250 | 5.316 | 2.293 | 6.63 |
| 82 | A | G | 0.098 | 4.215 | 0.000 | 9.17 |

TABLE 6
SNP markers in QTL regions 1 to 21: Differences (termed δ) in mean
percentage O/DM for oil palm plants including a SNP allele associated
with the high-oil-production trait (termed Max) versus oil palm plants
lacking the SNP allele (termed Min), with respect to the genotype model
for the Nigerian x AVROS population and the Deli × AVROS population.
SNP numbering is in accordance with Table 3.

| | SNP effects (Genotype): | | | SNP effects (Genotype): | | |
|---|---|---|---|---|---|---|
| | Nigerian x AVROS | | | Deli x AVROS | | |
| | mean O/DM (%) | | | mean O/DM (%) | | |
| SNP No. | Min | Max | δ | Min | Max | δ |
| 1 | 75.31 | 76.60 | 1.29 | 75.91 | 76.95 | 1.04 |
| 2 | 75.07 | 75.91 | 0.84 | 76.47 | 76.93 | 0.46 |
| 3 | 75.21 | 75.51 | 0.30 | 75.98 | 76.95 | 0.97 |
| 4 | 75.33 | 77.19 | 1.86 | 76.50 | 76.93 | 0.44 |
| 5 | 74.21 | 75.72 | 1.50 | 74.69 | 76.84 | 2.15 |
| 6 | 74.96 | 75.54 | 0.59 | 76.70 | 77.11 | 0.41 |
| 7 | 73.46 | 75.73 | 2.27 | 75.82 | 76.99 | 1.18 |
| 8 | 74.77 | 75.98 | 1.20 | 76.37 | 77.51 | 1.14 |
| 9 | 75.20 | 76.45 | 1.25 | 75.98 | 76.99 | 1.00 |
| 10 | 74.88 | 75.79 | 0.91 | 76.37 | 77.38 | 1.00 |
| 11 | 73.56 | 75.60 | 2.04 | 74.65 | 76.99 | 2.34 |
| 12 | 75.29 | 76.17 | 0.89 | 76.29 | 77.38 | 1.09 |
| 13 | 75.16 | 75.74 | 0.58 | 74.65 | 77.01 | 2.36 |
| 14 | 74.73 | 75.58 | 0.86 | 74.95 | 77.40 | 2.45 |
| 15 | 74.68 | 76.13 | 1.45 | 76.29 | 77.23 | 0.94 |
| 16 | 74.98 | 76.10 | 1.12 | 76.19 | 77.18 | 0.99 |
| 17 | 73.80 | 75.59 | 1.79 | 75.83 | 77.00 | 1.17 |
| 18 | 73.00 | 75.48 | 2.48 | 75.50 | 77.04 | 1.54 |
| 19 | 73.22 | 75.99 | 2.77 | 76.19 | 77.18 | 0.98 |
| 20 | 73.71 | 77.80 | 4.09 | 74.76 | 79.80 | 5.04 |
| 21 | 74.90 | 75.91 | 1.02 | 76.19 | 77.26 | 1.07 |
| 22 | 74.53 | 75.77 | 1.24 | 76.14 | 77.19 | 1.05 |
| 23 | 74.91 | 76.32 | 1.41 | 69.80 | 77.21 | 7.41 |
| 24 | 74.69 | 75.77 | 1.08 | 76.14 | 77.21 | 1.08 |
| 25 | 75.34 | 75.48 | 0.14 | 75.82 | 77.01 | 1.19 |
| 26 | 74.89 | 75.92 | 1.04 | 76.07 | 76.99 | 0.92 |
| 27 | 73.16 | 75.98 | 2.82 | 76.05 | 77.23 | 1.18 |
| 28 | 75.16 | 75.62 | 0.46 | 75.83 | 77.40 | 1.56 |
| 29 | 75.22 | 76.25 | 1.03 | 76.05 | 76.96 | 0.92 |
| 30 | 75.24 | 75.73 | 0.49 | 76.05 | 76.96 | 0.91 |
| 31 | 74.86 | 75.58 | 0.71 | 74.85 | 77.01 | 2.16 |
| 32 | 74.84 | 75.60 | 0.76 | 76.01 | 77.24 | 1.23 |
| 33 | 74.84 | 75.60 | 0.76 | 73.25 | 77.24 | 3.99 |
| 34 | 75.19 | 75.65 | 0.46 | 73.25 | 77.26 | 4.01 |
| 35 | 75.19 | 75.65 | 0.46 | 73.25 | 77.26 | 4.01 |
| 36 | 74.76 | 78.50 | 3.74 | 76.01 | 77.24 | 1.23 |
| 37 | 74.22 | 76.34 | 2.12 | 76.10 | 77.19 | 1.09 |
| 38 | 74.32 | 75.77 | 1.45 | 69.80 | 77.20 | 7.40 |
| 39 | 74.48 | 75.81 | 1.33 | 75.53 | 76.88 | 1.35 |
| 40 | 74.48 | 75.81 | 1.33 | 76.46 | 76.87 | 0.41 |
| 41 | 74.48 | 75.81 | 1.33 | 74.47 | 76.88 | 2.41 |
| 42 | 74.76 | 75.73 | 0.97 | 73.90 | 76.88 | 2.98 |
| 43 | 74.19 | 75.80 | 1.61 | 73.25 | 77.22 | 3.97 |
| 44 | 74.16 | 75.88 | 1.72 | 76.25 | 76.95 | 0.70 |
| 45 | 74.38 | 75.64 | 1.26 | 73.25 | 77.05 | 3.80 |
| 46 | 73.81 | 76.37 | 2.56 | 69.80 | 76.98 | 7.18 |
| 47 | 74.87 | 75.76 | 0.89 | 75.88 | 76.97 | 1.10 |
| 48 | 74.09 | 75.77 | 1.69 | 73.25 | 77.18 | 3.93 |
| 49 | 74.51 | 75.77 | 1.26 | 76.33 | 76.97 | 0.65 |
| 50 | 74.82 | 76.22 | 1.40 | 75.68 | 77.23 | 1.56 |
| 51 | 75.11 | 75.56 | 0.45 | 74.65 | 77.01 | 2.36 |
| 52 | 74.40 | 76.07 | 1.67 | 75.79 | 77.01 | 1.22 |
| 53 | 74.54 | 75.80 | 1.27 | 75.92 | 76.99 | 1.07 |
| 54 | 74.71 | 75.67 | 0.96 | 73.25 | 77.27 | 4.02 |
| 55 | 74.90 | 75.72 | 0.82 | 76.61 | 76.98 | 0.38 |
| 56 | 74.90 | 75.72 | 0.82 | 76.59 | 77.17 | 0.58 |
| 57 | 74.91 | 75.73 | 0.82 | 76.65 | 76.97 | 0.32 |
| 58 | 73.64 | 76.13 | 2.49 | 75.82 | 77.00 | 1.18 |
| 59 | 75.00 | 75.67 | 0.68 | 75.82 | 77.00 | 1.18 |
| 60 | 73.80 | 76.01 | 2.22 | 75.82 | 76.99 | 1.17 |
| 61 | 74.91 | 77.34 | 2.43 | 76.10 | 79.90 | 3.80 |
| 62 | 74.63 | 75.74 | 1.11 | 76.19 | 76.98 | 0.79 |
| 63 | 74.63 | 75.74 | 1.11 | 75.97 | 76.98 | 1.01 |
| 64 | 74.63 | 75.74 | 1.11 | 75.97 | 76.98 | 1.01 |
| 65 | 74.87 | 75.96 | 1.10 | 76.45 | 76.84 | 0.39 |
| 66 | 75.31 | 76.05 | 0.74 | 73.25 | 76.97 | 3.72 |
| 67 | 75.41 | 75.41 | 0.00 | 76.59 | 79.38 | 2.79 |
| 68 | 73.39 | 75.59 | 2.20 | 76.10 | 78.74 | 2.64 |
| 69 | 75.00 | 76.18 | 1.18 | 75.16 | 77.08 | 1.92 |
| 70 | 75.00 | 76.18 | 1.18 | 75.16 | 77.08 | 1.92 |
| 71 | 75.41 | 75.41 | 0.00 | 75.31 | 77.63 | 2.33 |
| 72 | 75.41 | 75.41 | 0.00 | 74.55 | 77.09 | 2.54 |
| 73 | 75.30 | 75.53 | 0.24 | 76.54 | 77.81 | 1.28 |
| 74 | 75.23 | 76.07 | 0.85 | 76.56 | 78.38 | 1.82 |
| 75 | 75.23 | 76.07 | 0.85 | 76.51 | 78.49 | 1.98 |
| 76 | 73.69 | 75.60 | 1.91 | 76.60 | 78.30 | 1.70 |
| 77 | 73.69 | 75.60 | 1.91 | 76.57 | 78.51 | 1.94 |
| 78 | 73.69 | 75.65 | 1.96 | 76.60 | 78.30 | 1.70 |
| 79 | 73.69 | 75.60 | 1.91 | 76.57 | 78.51 | 1.94 |
| 80 | 74.95 | 75.75 | 0.80 | 76.60 | 78.30 | 1.70 |
| 81 | 73.69 | 75.60 | 1.91 | 76.57 | 78.51 | 1.94 |
| 82 | 74.60 | 75.89 | 1.29 | 74.15 | 77.01 | 2.86 |

### INDUSTRIAL APPLICABILITY

The methods disclosed herein are useful for predicting oil yield of a test oil palm plant, and thus for improving commercial production of palm oil.

1. A method for predicting palm oil yield of a test oil palm plant, the method comprising the steps of:
(i) determining, from a sample of a test oil palm plant or a population of oil palm plants, at least a first single nucleotide polymorphism (SNP) genotype of the test oil palm plant, the first SNP genotype corresponding to a first SNP marker, the first SNP marker (a) being located in a first quantitative trait locus (QTL) for a high-oil-production trait and (b) being associated, after stratification and kinship correction, with the high-oil-production trait with a genome-wide −log₁₀(p-value) of at least 4.0 in the population or having a linkage disequilibrium r² value of at least 0.2 with respect to a first other SNP marker that is linked thereto and associated, after stratification and kinship correction, with the high-oil-production trait with a genome-wide −log₁₀ (p-value) of at least 4.0 in the population;
(ii) comparing the first SNP genotype of the test oil palm plant to a corresponding first reference SNP genotype indicative of the high-oil-production trait in the same genetic background as the population; and
(iii) predicting palm oil yield of the test oil palm plant based on the extent to which the first SNP genotype of the test oil palm plant matches the corresponding first reference SNP genotype,
wherein the first QTL is a region of the oil palm genome corresponding to one of;
(1) QTL region 1, extending from nucleotide 66542323 to 66776312 of chromosome 1;
(2) QTL region 2, extending from nucleotide 66807385 to 67299617 of chromosome 1;
(3) QTL region 3, extending from nucleotide 62277032 to 62355782 of chromosome 2;
(4) QTL region 4, extending from nucleotide 31132787 to 31173962 of chromosome 4;
(5) QTL region 5, extending from nucleotide 32863621 to 32964104 of chromosome 5;
(6) QTL region 6, extending from nucleotide 33355931 to 33509217 of chromosome 5;
(7) QTL region 7, extending from nucleotide 33658904 to 34233352 of chromosome 5;
(8) QTL region 8, extending from nucleotide 34358119 to 34997228 of chromosome 5;
(9) QTL region 9, extending front nucleotide 35004388 to 35125743 of chromosome 5;
(10) QTL region 10, extending from nucleotide 35191678 to 35193677 of chromosome 5;
(11) QTL region 11, extending from nucleotide 36108847 to 36272808 of chromosome 5;
(12) QTL region 12, extending from nucleotide 39210662 to 39225076 of chromosome 5;
(13) QTL region 13, extending from nucleotide 39518005 to 40469897 of chromosome 5;
(14) QTL region 14, extending from nucleotide 40535309 to 40690150 of chromosome 5;
(15) QTL region 15, extending from nucleotide 40789706 to 40983955 of chromosome 5;
(16) QTL region 16, extending from nucleotide 41001085 to 41302446 of chromosome 5;
(17) QTL region 17, extending from nucleotide 3050807 to 3241977 of chromosome 8;
(18) QTL region 18, extending from nucleotide 5354764 to 5445890 of chromosome 8;
(19) QTL region 19, extending front nucleotide 29488933 to 29602300 of chromosome 9;
(20) QTL region 20, extending front nucleotide 4797284 to 5717606 of chromosome 11; or
(21) QTL region 21, extending front nucleotide 8611715 to 8857914 of chromosome 15.

2. The method of claim 1, wherein the high-oil-production trait comprises increased oil-to-dry mesocarp.

3. The method of claim 1 or 2, wherein the population of oil palm plants comprises a Nigerian dura x AVROS pisifera population, a Deli dura x AVROS pisifera population, or a combination thereof.

4. The method of claims 1, 2, or 3, wherein:
the population of oil palm plants comprises a Nigerian dura x AVROS pisifera population;
the first QTL corresponds to one of QTL regions 2, 3, 8, 10, 13, 14, 16, 17, or 18; and
step (iii) further comprises applying a genotype model, thereby predicting the palm oil yield of the test oil paten plant.

5. The method of claims 1, 2, or 3, wherein:
the population of oil palm plants comprises a Nigerian dura x AVROS pisifera population;
the first QTL corresponds to one of QTL regions 3, 8, 10, 13. 15, 16, 17 or 18; and
step (iii) further comprises applying a dominant model, thereby predicting the palm oil yield of the test oil palm plant.

6. The method of claims 1, 2, or 3, wherein:
the population or oil palm plants comprises a Nigerian dura x AVROS pisifera population:
the first QTL corresponds to one of QTL regions 3, 4, 6, 7, 8, 9, 10, 11, 12, 13, 14, 16, 20, or 21; and
step (iii) further comprises applying a recessive model, thereby predicting the palm oil yield of the test oil palm plant.

7. The method of claims 1, 2, or 3, wherein:
the population of oil palm plants comprises a Deli dura x AVROS pisifera population;
the first QTL corresponds to one of QTL regions 1, 2, 4, 5, 6, 7, 8, 9, 11, 12, 13, 15, 16, 19, 20, or 21; and
step (iii) further comprises applying a genotype model, thereby predicting the palm oil yield of the test oil palm plant.

8. The method of claim 1, 2, or 3, wherein:
the population of oil palm plants comprises a Deli dura x AVROS pisifera population;
the first QTL corresponds to one of QTL regions 8, 10, or 13; and
step (iii) further comprises applying a dominant model, thereby predicting the palm oil yield of the test oil palm plant.

9. The method of claims 1, 2, or 3, wherein:
the population at oil palm plants comprises a Deli dura x AVROS pisifera population;
the first QTL corresponds to one of QTL regions 1, 2, 4, 5, 6, 7, 8, 9, 11, 12, 13, 15, 16, 19, 20, or 21; and
step (iii) further comprises applying a recessive model, thereby predicting the palm oil yield of the test oil palm plant.

10. The method cat any one of claim 1-9, wherein the test oil palm plant is a tenera candidate agricultural production plant.

11. The method of claims 1 or 2, wherein the population of oil palm plants comprises a Nigerian dura x Nigerian dura population, a Nigerian dura x Deli dura population, a Deli dura x Deli dura population, an AVROS pisifera x AVROS tenera population, an AVROS tenera x AVROS tenera population, or a combination thereof.

12. The method of claims 1, 2, or 11, wherein the test oil palm plant is a plant for mother palm selection and propagation, a plant for introgressed mother palm selection and propagation, or a plain for pollen donor selection and propagation.

13. The method of any one of claims 1-12, wherein the test oil palm plant is a seed, a seedling, a nursery phase plant, an immature phase plant, a cell culture plant a zygotic embryo culture plant, or a somatic tissue culture plant.

14. The method of any one of claims 1-12, wherein the test oil palm plant is a production phase plant, a mature palm, a mature mother paint, or a mature pollen donor.

15. The method of any one of claims 1-14, wherein:
step (i) further comprises determining, from the sample of the test oil palm plant, at least a second SNP genotype of the test oil palm plant, the second SNP genotype corresponding to a second SNP marker, the second SNP marker (a) being located in a second QTL for the high-oil-production trait and (b) being associated, after stratification and kinship correction, with the high-oil-production trait with a genome-wide −log₁₀(p-value) of at least 4.0 in the population or having a linkage disequilibrium r² value of at least 0.2 with respect to a second other SNP marker that is linked thereto and associated, after stratification and kinship correction, with the high-oil-production trait with a genome-wide −log₁₀(p-value) of at least 4.0 in the population; and
step (ii) further comprises comparing the second SNP genotype of the test oil palm plant to a corresponding second reference SNP genotype indicative of the high-oil-production trait in the same genetic background as the population,
wherein the second QTL corresponds to one of QTL regions 1 to 21, with the proviso thin the first QTL and the second QTL correspond to different QTL regions.

16. The method of claim 15, wherein step (iii) further comprises predicting palm oil yield of the test oil palm plant based on the extent to which the second SNP genotype of the test oil palm plant matches the corresponding second reference SNP genotype.

17. The method of claims 15 or 16, wherein:
step (i) further comprises determining, front the sample of the test oil palm plant, at least a third SNP genotype to a twenty-first SNP genotype of the test oil palm plant, the third SNP genotype to the twenty-first SNP genotype corresponding to a third SNP marker to a twenty-first SNP marker, respectively, the third SNP marker to the twenty-first SNP marker (a) being located in a third QTL to a twenty-first QTL, respectively, for the high-oil-production trait and (b) being associated, alter stratification and kinship correction, with the high-oil-production trait with a genome-wide −log₁₀(p-value) of at least 4.0 in the population or having linkage disequilibrium r² values of at least 0.2 with respect to a third other SNP marker to a twenty-first other SNP marker, respectively, that are linked thereto and associated, after stratification and kinship correction, with the high-oil-production trait with a genome-wide —log₁₀(p-value) of at least 4.0 in the population; and
step (ii) further comprises comparing the third SNP genotype to the twenty-first SNP genotype of test oil paint plant to a corresponding third reference SNP genotype to a corresponding twenty-first reference SNP genotype, respectively, indicative or the high-oil-production trait in the same genetic background as the population,
wherein the third QTL to the twenty-first QTL each correspond to one of QTL regions 1 to 21, with the proviso that the first QTL to the twenty-first QTL each correspond to different QTL regions.

18. The method of claim 17, wherein step (iii) further comprises predicting palm oil yield or the test oil palm plant based on the extent to which the third SNP genotype to the twenty-first SNP genotype of the test oil palm plant match the corresponding, third reference SNP genotype to the corresponding twenty-first reference SNP genotype, respectively.

19. A method of selecting a high-palm-oil-yielding oil palm plant for agricultural production of palm oil, the method comprising the steps of:
(a) predicting palm oil yield of a test oil paint plant according the method of any one of claims 1-18; and
(b) field planting the test oil palm plant for agricultural production of palm oil if the palm oil yield of the test oil palm plant is predicted to be higher than average for the population based on step (a).

20. A method of selecting a high-palm-oil-yielding oil palm plant for cultivation in cell culture, the method comprising the steps of:
(a) predicting palm oil yield of a test oil palm plant according the method or any one of claims 1-18; and
(b) subjecting at least one cell of the test oil palm plant to cultivation in cell culture if the palm oil yield of the test oil palm plant is predicted to be higher than average for the population based on step (a).

21. A method of selecting a parental oil palm plant for use in breeding to obtain agricultural production plants or improved parental oil palm plants, the method comprising the steps of:
(a) predicting palm oil yield of a test oil palm plant according the method of any one of claims 1-18; and
(b) selecting the test oil palm plant for use in breeding if the palm oil yield of tenera progeny of the test oil palm plant is predicted to be higher than average for the population based on step (a).

22. A SNP detection kit for predicting palm oil yield of a test oil palm plant, the kit comprising:
(i) a set of at least 21 nucleotide molecules suitable for determining, from a sample of a test oil palm plant of a population of oil palm plants, a first SNP genotype to a twenty-first SNP genotype, respectively, of the test oil palm plant, the first SNP genotype to the twenty-first SNP genotype corresponding to a first SNP marker to a twenty-first SNP marker, respectively, the first SNP marker to the twenty-first SNP marker (a) being located in a first QTL to a twenty-first QTL, respectively, for a high-oil-production trait in the population and (b) being associated, after stratification and kinship correction, with the high-oil-production trait with a genome-wide −log₁₀(p-value) of a least 4.0 in the population or having linkage disequilibrium r² values of at least 0.2 with respect to a first other SNP marker to a twenty-first other SNP marker, respectively, that are linked thereto and associated, after stratification and kinship correction, with the high-oil-production trait with a genome-wide −log₁₀(p-value) of at least 4.0 in the population; and
(ii) a reference sample of a reference high-oil-yielding oil palm plant of the population,
wherein the first QTL to the twenty-first QTL are regions of the oil palm genome corresponding, respectively, to:
(1) QTL region 1, extending from nucleotide 66542323 to 66776312 of chromosome 1;
(2) QTL region 2, extending from nucleotide 66807385 to 67299617 of chromosome 1;
(3) QTL region 3, extending from nucleotide 62277032 to 62355782 of chromosome 2;
(4) QTL region 4, extending from nucleotide 31132787 to 31173962 of chromosome 4,
(5) QTL region 5, extending from nucleotide 32863621 to 32964104 or chromosome 5;
(6) QTL region 6, extending from nucleotide 33355931 to 33509217 of chromosome 5;
(7) QTL region 7, extending from nucleotide 33658904 to 34233352 of chromosome 5;
(8) QTL region 8, extending from nucleotide 34358119 to 34997228 of chromosome 5;
(9) QTL region 9, extending from nucleotide 35004388 to 35125743 of chromosome 5;
(10) QTL region 10, extending from nucleotide 35191678 to 35193677 of chromosome 5;
(11) QTL region 11, extending from nucleotide 36108847 to 36272808 of chromosome 5;
(12) QTL region 12, extending from nucleotide 39210662 to 39225076 of chromosome 5;
(13) QTL region 13, extending from nucleotide 39518005 to 40469897 of chromosome 5;
(14) QTL region 14, extending from nucleotide 40535309 to 40690150 or chromosome 3;
(15) QTL region 15, extending from nucleotide 40789706 to 40983955 of chromosome 5;
(16) QTL region 16, extending from nucleotide 41001085 to 41302446 of chromosome 5;
(17) QTL region 17, extending from nucleotide 3050807 to 3241977) of chromosome 8;
(18) QTL region 18, extending from nucleotide 5354764 to 5445890 of chromosome 8;
(19) QTL region 19, extending from nucleotide 29488933 to 29602300) of chromosome 9;
(20) QTL region 20, extending from nucleotide 4797284 to 5717606 of chromosome 11; and
(21) QTL region 21, extending from nucleotide 8611715 to 8857914 of chromosome 15;

23. The SNP detection kit of claim 22, further comprising a solid substrate, the nucleotide molecules being attached to the solid substrate.

24. The SNP detection kit of claim 22 or 23, wherein the nucleotide molecules are oligonucleotide or polynucleotides.


SEQUENCE LISTING (42 sequences)

SEQ ID NO: 1 - DNA, 500 bp, Elaeis guineensis
  misc_feature (1)..(500): Elaeis guineensis linkage group LG1 66542323 to 66542822
gagaagaggaagcgtgcaggacggagtatgcgtgttgaaatttcgtgcagcccaacaggtccgagtctccaatactcctactctctaggctggtggtaagcaaagtttgggccctaccagaccctaccggactaatcagcattacctgcctctccttggtaccaagcatacctattcactaccttccgctgcgacaaacaagcaagtaaatcatcactggcgccatacttgatccgccattgttattgttaactatcgccatcatcactaatggccacacatattggccatgacactgggccctccggagcttcatgtgcattaatgggttggcttgaattagaatggtagttatcatagtaaatatggcataatctgtggtcaccctcaagatctggatgtctgcgtccaaaatctcacgaggtctgaggattggaacaaaacaaccaagtcagattgggttatccccatatttataagaactcaaaaagttattggag

SEQ ID NO: 2 - DNA, 500 bp, Elaeis guineensis
  misc_feature (1)..(500): Elaeis guineensis linkage group LG1 66775813 to 66776312
acacacacacttgggctaattaaaatacaaattctataactccaagaaatatattgaatgaaatattttcttgatttagttagttaaatacacaatgtcttacataagttagttgtgcctaggctggatcaaatttgatcagactaacccaagtttgtccatcaggttacattagacaatgcccatttaacacccaccaatttcagtagtttaagatcaaagcaattcagcaagagtcaatccaaacctggcataaccaaagcctggccataaaaccctaatttgacacaaactatacatgaagtcctcttctctattttttgggaagaaaaaaagaaaagaaaagaaaagggaatgctaggatcatgtagctaaatttaatttagcagaattatttttatttcaatttataggtaatgcttgatggtaaccttctgctagacttctctttgtgacgagagcaatggaatgatctaggattagattaagactcttttagt

SEQ ID NO: 3 - DNA, 500 bp, Elaeis guineensis
  misc_feature (1)..(500): Elaeis guineensis linkage group LG1 66807385 to 66807884
tggaaaaccttatacaactagtctgatctttgaccatcatagataaatttgctatacctcttcacatttcttaggttgttgttctcactattacttgcttctccacttcctgctcatcatactaatttcaaacagatttctccaagtagagaactgataatatgtgcagttgttgtaggctattctagctgcatcatctaagtgaaatatcaaacccttctataattagtatgtaattttgttttctctttcaacttatagttttacaggacatcttattttagacccatggctgttgatgtaatgtgagagtgactgtaagtgccgcatgggacactattttaggcaccatatggtatgtgcataagaaaataaagttgaaagtagatgccttctcttaagcagaaggttaatgagctaatagttccgtactaaatttatttcatatattattataatttttgtagaggaatatttgtcttaaccatcagatagcaatt

SEQ ID NO: 4 - DNA, 500 bp, Elaeis guineensis
  misc_feature (1)..(500): Elaeis guineensis linkage group LG1 67299118 to 67299617
aaagaatcgaaaaactttttctagtccacataattttcttgctaacattgaatgtcatcttcttcccgtgcccaccatattctgtctgtcagtttttgatcaccgacatgatccaccagagacttcaaagtggcacaaatagttaccaagagaattagtaaagttctttctcagtccacaagactccctctaagattgtacgtctcctcctcctatttccatcacattctatcagtttccgatgacacaatctaccgggatatttggtaatatcgtatatagttataagaagatagtaaaacttccttctataaatacatgcaggttgttgctaagattggttgtcgcctttttttcccatgatcatcaacacaatctaccgtgatctttgaggatagttctcaatccatttgaaaatgctgtttacatttattattgtacgttgcccgcctttagttgactagctattcaatcatgtgctgctacaactctgatccttg

SEQ ID NO: 5 - DNA, 500 bp, Elaeis guineensis
  misc_feature (1)..(500): Elaeis guineensis linkage group LG2 62277032 to 62277531
atttgatcctggtcatgtaaaggtgcgactgtcttcacaaggtgtagagcagaataccttgattccaagtttatagatcgagacgtttatcaattattgtatttgagtcaattactgatagagataaaggttcttggacacaaataccgacatatatatttatttttgtattgtatgatttggtttctcatgattgatatttatttataattgccgacatgattagttatatattttgtaacatatatttgtacaaaatttcgatggtttttgaatggtttggattatgatttgagagtttttgaaaagaacataatatgatacattatttttctggcataattaaaatttaataaattgatgatcaagaaaaggggtttggtatagtcttgctcaactgggaataccctcatcatagatagaaacgtgataccgagaatagtctgccatggatagatacatgatactgagaataatctgccatcgatagatatatgata

SEQ ID NO: 6 - DNA, 500 bp, Elaeis guineensis
  misc_feature (1)..(500): Elaeis guineensis linkage group LG2 62355283 to 62355782
aatgaatataatttaaaccaaagattcgacccatgatcagctctaaccatgccatggttgcaccagaagcctccctcgttgctccacccacatctatgatcatggtgttccaacatttccctacaaatttatcatgtggacccccctccacctaagagccagcaagcatacttactaaccttggagtggcaagcaatcgttttttcacctcttactcaaccatctattattgctctctactctattatgccaaactacacatattttatgtatgatttttgctggataatctcagatgaggagcccgatacaactgtttggcatcactctttataaagcttcttctaactcacattaaagtaaacttcattttttacaattactaggtattttccttcatcagggtggttgattaaaaaagggtccaaagagtggtaaaataaactcgagcagcacttcagtcattagtttggcatctttttctgttactaaaagctatc

SEQ ID NO: 7 - DNA, 500 bp, Elaeis guineensis
  misc_feature (1)..(500): Elaeis guineensis linkage group LG4 31132787 to 31133286
atatggagtcttagcagcagcttataaatctgtaagaacattgactgttctttttttcttgttctttttttttttgcttttcttctcatactttctctcattgttctaagattttaattttaattcaaaaatgttgtttcttttatccaggactcagaaaaagaaagcatttttcacaaaatagactggtacagagtggtactggatgaagcacacataatcaaatcttcaaaaaccagagttgctcaatcagcctttgcattaacctcacactgtaggtggtgcctaacaggtacccctcttcaggtcagtttcttcattttttgctagatcactttaccaaatcacgttggctgatctacactttacatgctgcaaatagaaacagggtggacctccacctttttccccaagataaaaaaggatggccgacccttaagaattatttttatgtaattgattcaagcagtactatataagttttcagttagatctccaaa

SEQ ID NO: 8 - DNA, 500 bp, Elaeis guineensis
  misc_feature (1)..(500): Elaeis guineensis linkage group LG4 31173463 to 31173962
actttgaacctaagtcacaggcttgaaataaattcatagataagccagaagttactctggctccatgaagctcagaaatactaatatgatataaattatatgtatgcaagtttataagcaaaacactggatctaatctaatggttggtatagctttgatttggataatgaaagtattattaaagaaccttaaaccccaaactcctagctctttgaatcaattataacttaattcaagcttgatacaagatcaaatttatttcatcttgatttgagataatgaagttgacaaactagttctttcaaaaataagcttgatcaaagcataaatatttcaaacttgagctcaaaattcatgataaaaaataatttcaaggtaagcttgtctagactcaggcttcaccaagctcaacttgattacaaccctagtaatgatcaggggccaaaattgtttcagacatactttcctccatttctttcattaaggtgatagtctgcaca

SEQ ID NO: 9 - DNA, 500 bp, Elaeis guineensis
  misc_feature (1)..(500): Elaeis guineensis linkage group LG5 32863621 to 32864120
atctcttttttcacagaatccagtatataattatgaagagaaaaaattatgagtaaacattttgaagagatgatcgatagatcataatgatggacgatcacaccattctaatatggatacaatcaaaaaaatcagaatgcaaatattctgtgggtttgtgggcaaacatcatatcagatccaatcttttatatacttgataataaaagatacaatccaatttatcatgctatttatcttttaagagatataacaaatagatatcgcaataaaatggtgaagaaactttcaatacgctgaaacatattaggtgacctatagaagtggatccaccatatgcacaacggacaaaggacagcgaacccaatcttatcacgtagaaactatattagcttgatgcacaggaaatggtctcctgtcatcaattagaaacttgccatccaatgatccggctaaaaggacaaaagccaccaaccgatcttatcacgtagaaactatatt

SEQ ID NO: 10 - DNA, 500 bp, Elaeis guineensis
  misc_feature (1)..(500): Elaeis guineensis linkage group LG5 32963605 to 32964104
tatgttaggtaatgaaatctagaatttctcttgcctgataagatttcctaaacaacagaggatggttgcttaaagataaagatggagataacacatgccctgatttttaaaaacaaatatgaggggagagaaggaaagagaagagggtccacctagataagcatgatggaagggagataaacacagaaggagcaagattagatacaagagggagaaggaggtcgaggattaggaccacacacatacacatacagagagagaggactggcaggttggggggtttggagacaaaaggacagctattaggaaaatcataatgggtattttatcatatttgtaacaacaactgtgcaatagatttcttaataagacatatccaacaaacactgatggtaacttaggaactaaattgatggacccaaaaattcaatctaagatcttttcatatagcataggtatagtttcaaccggatgtgccttttactaatggatttcttgag

SEQ ID NO: 11 - DNA, 500 bp, Elaeis guineensis
  misc_feature (1)..(500): Elaeis guineensis linkage group LG5 33355931 to 33356430
cagcatcataacaaaaccatggattctacagtagtagaaatagagacctcatgctctgattcctttactcattaattgctcaggtgatcatagcccaaaggcagctcggtcaaacacacacaaagtaggagttaaaagtaatggaaatatagaaatgagcatgggggaggttgcaatgatagtatacatgtgataatctcagacaatcaataacttgtacaagtacagtaagtgaagtttccaacttatgtaacaacattacaccaataagaaacttatgaagaaaatattattgcacaaggaggtattcagcctttcaggtgaggaattaagagacccctcgggaatgaatacgtagcagtgcacccatggtgccatttgcgagggaccaccccacccacctttgacccagggatgggagatcttgaggccgccggacgaaggcgtgcacggggcccggcgcgcgtgccggaccggtgtcagagcccgcaatcatgcgt

SEQ ID NO: 12 - DNA, 500 bp, Elaeis guineensis
  misc_feature (1)..(500): Elaeis guineensis linkage group LG5 33508718 to 33509217
tgctttctaacttcgaggtttagatcttatcatgcttagaaaaattagtttgttgtttaaaaaattataatcttctgctgtttcacactgaagctcacatcaggctttccccaccagcatcatagctgcaactgcatcctaataagactttctttctacccaaagctactagtatgtccaccaaaaattgcaaatatttaacttactataaatcataaatttacatgatcttatccaaaaaaataaaagaaccacattccagcatattctatacaagtataaggatgtgccagaaaaacttgcctccatgtgacgagaaggataagatggtatatttcctatactgttcccacttgataatgtttgtgatgaactactcgaggagggccatgacaatcttgagaaaaatgatgttacaatgtctggaaactgcacaatgtacaaatactcagttgtcatatggaggaggaagatcttgccccacaataaaaagagacata

SEQ ID NO: 13 - DNA, 500 bp, Elaeis guineensis
  misc_feature (1)..(500): Elaeis guineensis linkage group LG5 33658904 to 33659403
tgaacctggtggttcatactgtatcgtgataaagtggtcaagaaccagaatggcttgggcagccggtcagttcagccctttaaattctcccaatccccccgcttggccccctcgagccccatttcgggtcctctctccctccctctctccctctctcatcttgatcgctctctctccatggattccataacgacttatacgatcacacgaggccaagcaactccatcaatccctagctccttcccctgccatgcttccaattccattgaaattcataaaacaaggatgcgcccaatccacgatccccagaccttctctactgtccacgacctcctccaccattagatccatgctccccaagccttctccatcatccacagcctcctccaccaccaaatccatgctcctatagcctcctccactgccagatccacactcctcatgcctccatcgtctacggcctcctccatcatccacaacctcctctgccaccaaatcca

SEQ ID NO: 14 - DNA, 500 bp, Elaeis guineensis
  misc_feature (1)..(500): Elaeis guineensis linkage group LG5 34232853 to 34233352
ttcattggagttcttaaatgcttctacagccaaacgtccaacttccatgactcgctcgttagccttctcgacctcagctacgcttgagaccatctcccaccgcaactcttgctcggaggtctccacctttctcagggcctctgtagcaactttcaccttttccttcacctcgattgcctgagatttggtcttctataactcttctttacgagtgagagcggagtctgatctacctaatgactccttcaggcacccaacttctgcctccaccgtctctgctcgggcacggacttattctgtttcgtccgcacgatttgataactcgacaagaaggtgaaacatctacccagaaactatgtaagaaagaggagaagagaggaatcagaaaatcaaagcatcatcacagtacctcatggagagaggctacggtgcgcttctataggctgacaaagctttgcctcctaaactttccttcctctaaaggaataagtatcccttcc

SEQ ID NO: 15 - DNA, 500 bp, Elaeis guineensis
  misc_feature (1)..(500): Elaeis guineensis linkage group LG5 34358119 to 34358618
agctataagtatccacctaacaatgctgtttccttaaaaaatattccggcggatgtggaaaagcatcaagtcgttgaggcaacatatcgtgcggaagagttccaagcttcttcttcttcttttttttttttttttttttttgaagcgaaccagttgaaagctatgttttgcaatgagtatttctaattaaatataaaaaatatttggttggaaagagtaaagatctgaaattagaatcagaatggatgactcccattccatccgtttagttgggaggagtcccattccgatttcgatttcgggatggaatgaaaatgagtcaatctatatagaactcaatctctactctcttttatagattcaatttttcatttcaattttgattttgattctgatttcgatcacaaaccaaacattttggaggatttagccatttcgattttgattccaaaccattccgatttttattttcattctgatttcgattacaaaccaaacat

SEQ ID NO: 16 - DNA, 500 bp, Elaeis guineensis
  misc_feature (1)..(500): Elaeis guineensis linkage group LG5 34996729 to 34997228
ccaaagactgagaggagatttttggatatttattaaattgcacggcatgtttcaaaaaaaaatatcttgttacatattattgttatatggtaaactgagaggagatttttggatatttattaaattgcatggcatgtttcaaaaaaaaatatcttgttacatattattgttatatggttatctttctgataattagttatgcttcagagatgttggtaatttactgagtccataaaactcatatatttttttatttttttagatacagaagaattttattggacttgggatcgaacagtgaggtgcatagcaggcctgtcatattctttagaatcttgtcttaagaagtatgcagccattacatgtccgatctgaatgttggatttgagacgtgacagtaataaataataaaatgttatcaagttattcaattttttttttactaggtgaatatgtgaaattgaaatgaggaagccatttggttttagtggtccgtgagt

SEQ ID NO: 17 - DNA, 500 bp, Elaeis guineensis
  misc_feature (1)..(500): Elaeis guineensis linkage group LG5 35004388 to 35004887
gaagacctgatttatagccctgatgtatttttttgatatctgtactgctgcccagacctggaccagaaccgatctttggatgtgtgggtcctgatttttggattagtaaggttggcaaaatttaaacccaaatccccacttacagttctgggtcaagatggaagtacctgaacccgacctgacctaatatcacccctaatcgcgaaacaccacaattaatgtgaagatgcaatttatttcttcacacattgcgacagttgaagacaatctaacttctgatcactcttagcttttaacctacgtggccttccttgttgatctggcatatatttgtcattatctttgtctacaaaatgcaattgtttattgaaaaaaagatttactcgtggatattaagtaggggagaatgaagctaccttattggaagatttgagcttttgctctttaaccttctttttctactgtcttaaggcttgcattagttgacgaataggcatgtg

SEQ ID NO: 18 - DNA, 500 bp, Elaeis guineensis
  misc_feature (1)..(500): Elaeis guineensis linkage group LG5 35125244 to 35125743
ctttggtggtgttgggactctgccgggttgttgctccaacacccaaggttattgctcacagccttcaggtcaactttgcctttcttttgaatcttttcttctttcttctaaaatcttctatcttctttagagtaattctcatggctagggcttctccttctcgaaaagatcgatcggagaatccgactgatgagtcccaatcgagtccagatgtggagtcctcttcactttcggaaccgaatgttgaacggctccgagagcagttttgtatcccggggcagttttaacttttcgcctccggagccgatggtcgggtgaataacccactttcggactaggtggccttctatgtcgaagatcttcggatggatcttcgattttcgattccgaagtttgtccgaaatattttggattattacggactttgcccggcttagctggcaccgaactctgtccaactgataattagctttgccttgctgcgtcggatgttgccgacc

SEQ ID NO: 19 - DNA, 500 bp, Elaeis guineensis
  misc_feature (1)..(500): Elaeis guineensis linkage group LG5 35191678 to 35192177
ttttgtggtgtaagatattgtaaggtatcaagcatgacattggtatatgatgtcagaaggccattaatacagtttgttttggtaatgaaatgttgtgccttgttaggaagatttgactattataattttcaggtaaaattattgctatgatatttttatagccttactaatattctaaatattcataatttgtgtataatttcaattctcatagaaggcagcaccatttctaaacatttaactaaattgtagacttgctgttgaaacatgctaagaatatgttctttcaattggtttgatgatcgctcggcataacccttagaataggttacaaaaggtacgatatagacctttcatgcatagcatgtaatattcggccttgctaggataggttacagaaagtaggatatagacctttcatgcatagcaggtaacattctgccttgttttttttgtgatagttcgaccctaagtcctggagagaatttaccggatggagg

SEQ ID NO: 20 - DNA, 500 bp, Elaeis guineensis
  misc_feature (1)..(500): Elaeis guineensis linkage group LG5 35193178 to 35193677
gccatgagtgattcatgaaaatttaattaatatatattttgtggcatgctatgtgattgttaatcatggtcaaagggagtaattacctctgctcataatcgaaagagcattatttttgagattcctttttaagggaaagaaacccaactttccgttgttatttgctcataatctgttgtctggattcacactaccttcattatcagtcagttctctaccatttagactctttttttgcatgagttacttatctttattcttactttaagcacttgctgaagagtttactgatggttcatgtaaattgtttcaggctttgcattttcccttttaaactttccagcctcatatagatacggcaaaacaatcaaattctttgtcaatggttgcaagagagatctagtggcacaaacttggtgtcatatggggcctgtcattgatcccattttgttggaaggttggatctaattagacatgtatctaaataaggatcttaggat

SEQ ID NO: 21 - DNA, 500 bp, Elaeis guineensis
  misc_feature (1)..(500): Elaeis guineensis linkage group LG5 36108847 to 36109346
gtgaaggggcaatattcaggtttcgactatcgaatcggaacctacagttaggagaaatgaagatcccactccgatcgtcaagcctgcgatttccttgccagcgatcgtgggggtcgcactcgaggagaacccatcaaagaaaatattgatggagcaagcacagagagaggatgcgagcaaccacccatcaggagcttctgcaaaggagatacgagtggagagcacgatcgatctcctcgaagggatgctggaagcgggcatggagactgatgagtcccaagagctggtgatggctgcccgatgtacccctatggctatcctccatgtgggtcccttgcagtcggcgtcttctgctccctcagtgatagaggatctccaagccatggcaagactcctgagggattttcttcctccattcaagaggcagcctttggatggacgaggagtgcagcagtaaatgttgggcgcccttagatatctcatccaggtttaaggtctcc

SEQ ID NO: 22 - DNA, 500 bp, Elaeis guineensis
  misc_feature (1)..(500): Elaeis guineensis linkage group LG5 36272309 to 36272808
gagacttctaaggctttagaaggtgttttataaacttgatagttttctatccatatttaaccagagtgagttaatttttataaaaactaacaatagtggctgatttgaatgttaccttaaaattatagtgactattatagaacaaaagaaaaaggtttctaacctgtaatttctaaaaaggtataatgattgtcctgcaaaaaaaatccttctaaatactttctgcctccgaaaatagctcaaaagcatcctgccaataccccaattccaattttaactaaataaaagatcattaagagaagcttacaaactaaaatgcaaatgattgctccatgtcgaaccttcttttttcttctaaaagattcctattagatattaaacaaaataaaggagaagatggttcagtgaattaggatttttgttatttgttataatatatttaatgtttataacaagtagttagttgttggaatcttgtgaatagtggatagctgacaggc

SEQ ID NO: 23 - DNA, 500 bp, Elaeis guineensis
  misc_feature (1)..(500): Elaeis guineensis linkage group LG5 39210662 to 39211161
cggtgggttgttgatgcactcctccatgaacgccagcaagtcatcatgatccttactagtgtaatccttaacccgcttgtccttccattcaccagatcgagccaaagattgcgcttccatgtttgatgatgttttcttcttggaggagagaactagcaacctagcttgatcatcaaggtactcagattgcatgttggcttgttcttcaatatggtttgaactgtcaagcagtgctcgtactcgcttgttggtgaccgtatcctgttgtaaaccattgatttctctcaagcactttgtatcatcatgaactatagaatgagatgttgattcagttgcttggtcctcatatatagctccaccatcttttgtattcttttctctattgttcaagtaaactttacaaataacccatgtatcaagctgccaaaagatgtgagataagagaactaaattatgatgtaattcatgactataagatattagatgtgattatttggatg

SEQ ID NO: 24 - DNA, 500 bp, Elaeis guineensis
  misc_feature (1)..(500): Elaeis guineensis linkage group LG5 39224577 to 39225076
aatgtgacccattgttttagcgaaacaagctaaattttacattgatttgatttgcccatttttctgatttgcaaccaagtgagggctgaacctcactagccaaacctaggattttaaatgaatcctctaattcatatcatgcccaaaacaagtatttgggtcagcttgcttatgtgtttggttttgcccgggccattcgtgttctaattaaataattgcataaagtttccgacctaagcctggccttgagctccattttcttcatcttggtcgatcataaaatatctagatggatcatgtcacacgacccatttgcacctttatagccacaatcatgaaattgatgtacaactatagctctattttaccatcaccccatgtgaatagcaccaagcatctccaatctgcttcaatcattaatgtatttttcctaaaatttaaggagacatgcaacaacagtccttttgagcaagacttggaattaaaatccccaatttttc

SEQ ID NO: 25 - DNA, 500 bp, Elaeis guineensis
  misc_feature (1)..(500): Elaeis guineensis linkage group LG5 39518005 to 39518504
gagtagtaagaagcaaatatgtatagcattatccataatggaagctgaatttgtagcttgttcagcagtagtacaggaagccatctggttgaaaagatttcttaagcatttgggattcttgagcatggagaagctttgtgaacagaaccccaccgatcttgcttttctgtctgaaatctactcatctagtgccagtgcacgtaactctattcccttaggaaagcaggtcagcaagtgggattttaggtaaaaaagattcgttctatctggactctttctatagggtgtaacattcattcctggactatgaaaatgtaaatcctgaaagcgggcatcattgcatgccttccaggtctcgtaaaagatcaaacgagtaagaccttattcttcttaaatggatccatcctaaccgaaatcattgaattcggatccgactaaactagacttattgcttcgtcttttgcatgcttttgctatctgtctttccatccagctttgct

SEQ ID NO: 26 - DNA, 500 bp, Elaeis guineensis
  misc_feature (1)..(500): Elaeis guineensis linkage group LG5 40469398 to 40469897
ggtagggataggtagggaagtgttgacattgacacctttatcttgaagaaacttgtctaataaggaaatgtcttgcttctaacttacaagaggggttagtggaaactggtaaaaccttagcagcatgaaactcctacctaggttcccacctctactttgggatctttcaggaaacaagctcatgcaaccctttcccaagcacttcactttctccttttcttttaaccttttttcttgagagaaggagatccacttcgttgcacctgttctgctcctgctctaggtaacaaagagaagttctaagatccttattcattttgattatggatgattatacctagagtgctcttaagcaattaatttcatacagtagtattgcatttcaaataaaggaaccaaaaagtatgatggaagcataagaaagaaaatacctttcagttccatgtgattcttagtctcttcaccaagttgcttccacaaacctgtcaaaattgcacttg

SEQ ID NO: 27 - DNA, 500 bp, Elaeis guineensis
  misc_feature (1)..(500): Elaeis guineensis linkage group LG5 40535309 to 40535808
atacaaacctgggactaataatggcagagttcaacccaacccaaatccgaacccacaaaaatatatactatatatattagatacaatttcatcttccaagatcttactatgttacttgaaattgggttccacgtatattcacatgttgccattcctagcgcaatttgttttatttttagatataattcatatgacgggggaatttacacgtggctttttggtgacccaagccaatttttattggggtgggtcaggttggtttgggccaattagattagtttaaccaagtcacatgaggtcaaaaattgaggtcaggttgggttctggttgtgaaacttgggttaaagatcgtgttgggttttaggcctaggtctcgaggtgtcaggttaggtctaagctggcctccagcccaatccatcttgccccacctaatttccactcttagccaaaaatgggctggaaccagcctaaaattggattggtacgactctacaagattg

SEQ ID NO: 28 - DNA, 500 bp, Elaeis guineensis
  misc_feature (1)..(500): Elaeis guineensis linkage group LG5 40689651 to 40690150
gaggaaggcaaaggtaaaggtgtcagtggggaagggagagcagcaaatgtcttgcatctgctggtagacatggatggcttcttggggataacaacccaaagaatagtttcttataagattgttccatattctcatgccagtttcattgatgtggtagcgatggagagcggtggtgaggagttgggagtgcacctggattgatgggtgcctcctggtttgatgtttgaggagaagagagaggagggcgtgctcatgggttgggtgtggtgggaggagatgatgactcagggttgatagcttttggtgtagaatgacaagggagagggaggagtcataggaagttgttctcagggggaagggcgatctggttaacaattgcattgactggagggtgaatcaatcactcaccgttggctgtgggttggcttccttttgaatgaatggtaggtgattacatgggggagggatgtggcaagggagtgagtgcaatgggagagggc

SEQ ID NO: 29 - DNA, 500 bp, Elaeis guineensis
  misc_feature (1)..(500): Elaeis guineensis linkage group LG5 40789706 to 40790205
acgtatgaaaccaaaatgatgcacctacacttggaattctagccggccatttacaacatctcatatattcctatttgcatgcgacttaaaaatgtaatcacacattaaagattgagttctgcaaacgatttcatttccctttttattagatattttataatcccaaattttcattcccaactagttctacgtaatagtttttatgcatgtcatgttataatcatgttgtgaggtgtaaaaaaatttcctaatcttgagaaattgctatacatgtgttttactaaagagactgcacgtgtgcatcgtatactttgctgttggtgtcgaagctagctggccatattgtcatgttgatacgtatctcttgttgctatgtgtacgtggctaaacgtaacccatataaaacattgacttccaagtgtttgacccattccactacctgctatgccaagggtttatgatggcaacctttcactcttgatccactcccatgcatgatt

SEQ ID NO: 30 - DNA, 500 bp, Elaeis guineensis
  misc_feature (1)..(500): Elaeis guineensis linkage group LG5 40983456 to 40983955
gaatcaggacttttagtctataaataaggtcttctggccaacgttgtaatcaatttattggagttattgaaaaaaaaaatccaaagaagagagtttcttccctttatcgtttttctttgtttcatgcatcttcaacctctctttccttagaaatgtggccctagactgcatcagttggtatcagagtgtagcagatctgtgagcgaagattcttccctgcagtctggatgacgggtagtggtcgcaagaaaaaccaacctgcgaatcaggcgacggcagctcatgatgaaatcacgcaatgggaacggaacctgatgttggagtgtgaagattggaggagacaggtggcggatttgattgcgcgtcttatgcagttggaaacccgactggcaagctttgaggtgaatccagaagatcgcagatcaaaggatgaagcttcagattttgagaatcctttccataggattgctcccacacactggcttaaaggatgaggtgat

SEQ ID NO: 31 - DNA, 500 bp, Elaeis guineensis
  misc_feature (1)..(500): Elaeis guineensis linkage group LG5 41001085 to 41001584
ctgtcatggatcataaatattgtaaattgcacatcatatgttgtcctattggtggcatcatttcagcaacaaaattcatgaggacaacttgaagaaaatcttgataaatagataccattaatcttttcttacttttgtagagtgaaaaacttcaaaaatcttgattacgttatgaaactacaacactatagatcataaatattataaatcaaaccatcctgtttcatgttagctgacttaagcttggggcctctaagcgcccactttcagaagtcatgacatgattcaagatgattacaagatctgctgtggataagtttggccattttaagtagatctggatgaggtttcagagcaagatctgccccatccaagattaaattatgtagagtaaggatacaactttcttatatgatatccattttcagcatcgtgagaaccaaaataaaatgatagagcagtaatataagagtgagatcatccccaaaagattcgccgaa

SEQ ID NO: 32 - DNA, 500 bp, Elaeis guineensis
  misc_feature (1)..(500): Elaeis guineensis linkage group LG5 41301947 to 41302446
gaatgacttccaggtttaaaagtcgttgtcgagttatcatttcagctaaagattttccaagttacaaatttgaagatagcaagaaagaaagaagaaaagggcaaatataccaagaatgctctccaatattagatttgattatgagttgaagagacccaagggatcgccggaaagcgcccgccatccgtgtgcacttttctcctcctcctcctctgatccaatcagtagaaaggaaagcaaacaggggttaaatattatggaagaagggggaagatttcaagccctattcctcttcgctgttctactccccttaaaacctaatcctccgcggccggcgtccccgtgcctctgtccttctccgattccagaccccaaccaccggataccttcgaattctaaggccggtggtgcgagggggcaagcggcggtggtggcggtgctccagcgcccgcagaggagggacggcgggttgcgttcagtgcttggaaaggggaaagtcg

SEQ ID NO: 33 - DNA, 500 bp, Elaeis guineensis
  misc_feature (1)..(500): Elaeis guineensis linkage group LG8 3050807 to 3051306
gaaaatcgagagagaagactcctaactgaagtcctgttctttctcttgactcacatgcacaatgcatgacttgttttttttctttttgttcttctggacaggttattacaataatacaacaaaatatcaagcaatttttttaaattatttttttttaaacatgcggaggaagtgagaaatttctccaagaattttgttaaataaaaagattatcgaagaatttacaagggagtaatgttacatggaaagagagcaaagtctatggtctcgtggacaatggaagaaactggtgccttactaattacttatttattatagtaaacaaattttagttattaaacggtcgaattagtatcatatgtagcatataagtgattgatttttagttattatatttactgatagaaagagacaatcacagcagatttcagtggactagtctttgcacttgcgttggagacttggcactttctattttgcgaaattattcaccctcaagg

SEQ ID NO: 34 - DNA, 500 bp, Elaeis guineensis
  misc_feature (1)..(500): Elaeis guineensis linkage group LG8 3241478 to 3241977
caaaaaaagcaaaatagtttggtgcaggaacaactgactaaaccatattcataaaagcaaaaggggagggagagagagagagcgggggacgggaaaattaaaaaagaagtttaccatgacaccaaaaccgatgccggtgatgacgaggttctcaatgccgacggcggccagctcaagggtgcctacgtggccgatcagtgtctgcgtcaccgagcccagcgagaactggaacagcgacgttaggatcgccggcccggcgatgtaccacaccaacctcaactcccgccacgtgtcctcccaccaaggccgccttcccttctcttcctcctcctcgttcctcctcctcgtgagcagaggcagagaaggatccgccgtcggcggcgccattcgtttttgtcggcggtggcgcaactccgaagactctctcttcccaagaaagaatgagaataaataaaggattcttaaaagaataaataaagggtttttttaacatgcgcccc

SEQ ID NO: 35 - DNA, 500 bp, Elaeis guineensis
  misc_feature (1)..(500): Elaeis guineensis linkage group LG8 5354764 to 5355263
gaaaaattgatcccttatggggagatcggttcgtccccaggttggaggattaaaggaagattgatcgctctatctccaattaggattgatctagctcgaagtggatcgactctggtgcaaagcctctcaggtccgtccgcagtacatatagatgcaagattctatactttgatagataaaagctaataaaccccactttttatcagaaattttttgttttagaagattggttaggggctacggtcctaatatgtggtatcagggagagattgttggctatattctagtgcacacccttcgaggtctcatgttagatttatgtggctggtgatgtggcaacaattctcagaagatcgatcccttataggaggtcggctctgtagcgaaaattttgtgcaggggcaaaatagtaattttaaaactttttcaaaattactattttacagcggaattattaattaatctcattaattaatattaattaaccctacactatgatc

SEQ ID NO: 36 - DNA, 500 bp, Elaeis guineensis
  misc_feature (1)..(500): Elaeis guineensis linkage group LG8 5445391 to 5445890
ctgttctacagggttataggcttatagcttagattccttctttttaagtgcaaaagcgtgcatattttaactattaatattaattttctttttctttaaggtcctttgccgaagaagatgttggaaagcacagtgttggtgtgcaatcagtagcaggtgctgcaaatgcaagcaaaggtttagaagttgcagttgcaaatcttcaagactactgtaatggtattcctcacacgattactctctctactattttctgaactcacatttatagtaaattttaatatgtctatctatttcagaattggagaataggttgttggctcgctttgatatagcgtcgcaaaggcgagatttaagaccaatggcagaatgtgctaaaattttatctcaggtgcaaaaattttatgttttatcaatatagttaatggcatttcccatctaaaagaaagaattgctggaaaaagctaggatgaaagtgaaaaggaagaggaagaaacaat

SEQ ID NO: 37 - DNA, 500 bp, Elaeis guineensis
  misc_feature (1)..(500): Elaeis guineensis linkage group LG9 29488933 to 29489432
acatgcttcttcaggatttttttttttcaataaatacaatgctgtgacatatggcatgtaatgcatttttgttttattacttctaatcatctccaaaagcatatataacctgtttaaaattttttcatgtagctataagctgaaaagaagcagatcaacaattcatcatcccgttcatgtatctcatacatgactgggttgcatattgtgattactattatttattccccctctgtattgatttatgttggccattctaatttccatttggtattctagtgttcctgatgagcagttgtttcattgtaaaaagcatataatgatttgtgcaggtgactgtaaatatgtgcatcgatgcttaacttaccatgtaaacgtgcggttttttcctgtggtgacatataattaaacatcattgcttcaaggcgggcatgatttttgcttgtatcatgtcatacttctttgcactgtgggatattaaacaatgtgttcttgcagca

SEQ ID NO: 38 - DNA, 500 bp, Elaeis guineensis
  misc_feature (1)..(500): Elaeis guineensis linkage group LG9 29601801 to 29602300
ttcagagactattaaatcacatctcttcatataaaaatccaatataaaagtctcttgaataacgtgtcaggggcttgccacatgatgatgtcccaattcctttcatataaatcacttctcgcatgatcaaacaaaaacctcatttcatataaaaagtttattgttacataatccaagaaggagatactagtaagcatcaatatcatcttcaaattaatgaagcataattgcagggacccatgggtactcccctcggaggttgaccacaaccaaacccactcaattaagtcaccacaagcaattgcccggcaaatctaatgatccgaagaatctaatgccaagacaaattcttaggattaactccaattataattctctcttatcaacaaccccatccacccatccacactctgccaggtctttgtactaaatccgccgtacagtgacgcgacgagatgccaggatccttccatccctaaccgtaattactttccttcccg

SEQ ID NO: 39 - DNA, 500 bp, Elaeis guineensis
  misc_feature (1)..(500): Elaeis guineensis linkage group LG11 4797284 to 4797783
agacatgtcacccatcctgcaaccccttcatatcaagtgtacccatctcctttactttctcttcaataggagctttcctcttattaattcatcaactctatattatatatataaatttagtcatagtaattcattaaattgaattacatctccaatgtttatggtctaaaataaatgtctttagtgcaacccatttctttatttaagccaaaaaacaatggaatattttggttacaaactcttctatatttttatcttgaaactgccatcatagacaccaattctctttgcacaatcttatcaagacctcttgccttgattgtagtctatctatccctgttgagctaccatcttctggttcgatctaccagcccgggattctcctctccatgatgcctgctccctgtgcggagtagcctgtatgatcttggtcccccaacccctccctcttttgcttgcaaaacaacatctactagaacatggtagcacatatgcacata

SEQ ID NO: 40 - DNA, 500 bp, Elaeis guineensis
  misc_feature (1)..(500): Elaeis guineensis linkage group LG11 5717107 to 5717606
atacaattattcaaattatgaaaaaaaaaccaatgtcaacaaaatcaatttggagccaaccttgtctccctccgtcgcataagatactctggaagcatttgtcttgttgccaaccagtactgctttctcagtctcatgatatgcatgtaatgaatggtttatgtatgcaacagtgtggggatcaagataggttctttttcatgataggctactgaaaacacagattttatctcatgtatatatctatctttacatcaacagcattcacagtagtccacctttgatcatccatatcctccaaacccaacgaaccttgtgttcagatgaggatttcatgggtgtcaagtaaacacaagaaagaaaaaaaaaaagaaaaaagaggaaatgacacaaagtgtaatcatgttatactaccttatccaggcacagtgattagaaaagcagcttacaaccagttcacaaagaagagggcattgtgctagaagagtcgtgctgccagg

SEQ ID NO: 41 - DNA, 500 bp, Elaeis guineensis
  misc_feature (1)..(500): Elaeis guineensis linkage group LG15 8611715 to 8612214
tgtcgttggctttgtggatggccaccgggaagtcggagagcgaaagggcctcctcttcttcctccaaggtcacccatttctcctgctgttcttgggccatggtctgggtgacttctttcacctctttgggggaagttggatgggagatttataaggggggaggtgggggatttggaaggggggacaggtggagccttggagggaaggaaagcaaagggtgcaggcttgccatgttcttttagactcggacattcctgtgggcctgtgggccggccctttcatgctcctctttaggctgttcacattagccttattttattaagggtcggctatgacccaggttgggttaaatagtaggagaaccaaccataataatctatggaactctatgaaagtattcaaatatatatagatgaggaggttggttaggttagttgatgacagaaaactcttatgatgttgtttattattaggaaaagtgataggttatgtagacattg

SEQ ID NO: 42 - DNA, 500 bp, Elaeis guineensis
  misc_feature (1)..(500): Elaeis guineensis linkage group LG15 8857415 to 8857914
aaggggcatcccgaaatatttttattaatgccctgaaattatttgttataaactcttttactctctaaggagattatatttattaatctctattgaattcttatgaggcaaaattactcaaataatattagaggtggccagaagccatataccactcttatcttgcctctttttatctccacctagataatttggagtaaccacacaatccaacaccatgtgaatctcatcctttctctcaaacgtaagtctaaacttcggtgctctcatcacacttgcaaggcactacaatgtactaacataaatatcataaatatcatttttatttcttttctcttataagagaagggttatctaactctaaaatccaacatgtatggaaacaggaacaactttggagtggtcctcttattaggtggaccggatctactgtgctcctaggttgaaatcatcataggtggattcattttattcgagaatgaaattattcggtgaattcg